(19) United States
(12) Patent Application Publication
(10) Pub. No.: US 2011/0027239 A1
(43) Pub. Date: Feb. 3, 2011

(54) ADIPOSE-DERIVED STROMAL CELLS (ASC) AS DELIVERY TOOL FOR TREATMENT OF CANCER

(75) Inventor: Hyun Joon Paek, Mililani, HI (US)

Correspondence Address: Nixon Peabody LLP, P.O. Box 60610, Palo Alto, CA, 94306, US

(73) Assignee: TISSUE GENESIS, INC., Honolulu, HI (US)

(21) Appl. No.: 12/511,940

(22) Filed: Jul. 29, 2009

(51) Int. Cl. A61K 35/12; A61P 35/04

(52) U.S. Cl. 424/93.21

(57) ABSTRACT

The present invention generally relates to use of adult Adipose-derived stromal cells (ASC) and genetically engineered ASC for the treatment of cancer. In particular, the present invention generally relates, in part to a method for treating a subject with cancer comprising administering to the subject a composition comprising engineered ASCs which have been modified to express a gene encoding at least one anti-cancer agent. In some embodiments, an anti-cancer agent is a pro-apoptotic agent. In some embodiments an anti-cancer agent is an agent which inhibits the expression of an oncogene.

### FIELD OF THE INVENTION

The field of the invention generally relates to a method of drug delivery for the treatment of cancer in a subject.

### BACKGROUND OF THE INVENTION

A central issue in cancer chemotherapy is the severe toxic side effects of anticancer agents on healthy tissues, which invariably imposes dose reduction, treatment delay or even discontinuance of therapy (Fennelly (1995) Clin. Cancer Res. 1:575-582; Hanjani, et al. (2002) Gynecol. Oncol. 85:278-284; Kobayashi, et al. (2002) Chronobiol. Int. 19:237-251; Ross and Small (2002) J. Urol. 167:1952-1956; Markman, et al. (2002) J. Clin. Oncol. 20:2365-2369; Sehouli, et al. (2002) Gynecol. Oncol. 85:321-326). Cytotoxicity for healthy organs can be significantly diminished by employing a drug delivery system which targets cancer cells (Alvarez, et al. (2002) Expert. Opin. Biol. Ther. 2:409-417; Dass and Su (2001) Drug Deliv. 8:191-213; Kopecek, et al. (2001) J. Controlled Rel. 74:147-158; Kunath, et al. (2000) Eur. J. Pharm. Biopharm. 49:11-15; Minko, et al. (2001) Dis. Manag. Clin. Outcomes 3:48-54; Vasey, et al. (2002) J. Clin. Oncol. 20:1562-1569).

In order to increase the efficiency of tumor therapy, it is important to deliver anti-tumor therapies and toxic agents specifically to the tumor cells. Traditional chemotherapeutic agents are administered systemically to a subject and circulate the subject's systemic circulation to reach the tumor. Systemic administration of an anti-cancer drug may have undesirable side effects if the anti-cancer drug acts non-specifically on non-cancer cells, and may also result in only a small amount of the anti-cancer drug actually reaching the site of the tumor. Thus, in some instances systemic administration of anti-cancer drugs are limited partly due to inability of the drug to efficiently contact the tumor cells. In particular, delivery of anti-tumor agents to a tumor cell masses can be inefficient due to limited access of the anti-tumor agent to the cancer cells because of the dense cell mass of the tumor.

When a tumor-agent is delivered systemically to a subject, the anti-tumor drug may only reach part of the tumor mass, and may also be at a too low a concentration to be effective in killing cancer cells should it actually reach the tumor. Additionally, the natural process of angiogenesis which provides vascularization and nutrients to the tumor may causes a concomitant increase in size of the tumor and thus increasing tumor mass and the likelihood that a systematically administered drug will not access a significant portion of the cancer cells due to the tumor mass density.

In some instances an anti-tumor drug or chemotherapeutic agent have been directed to the site of the tumor and specifically targets cancer cells using cell-surface targeting moieties, also called tumor associated antigens, attached to the anti-tumor drug which specifically recognizes and binds to cell-surface markers expressed specifically on cancer cells. However, even these methods suffer from lack of effectiveness as the tumor associated antigens are not necessarily ubiquitous.

Accordingly, there is a need for more targeted tumor therapies.

### SUMMARY OF THE INVENTION

The present invention generally relates to use of adult Adipose-derived stromal cells (ASC) and genetically engineered ASC for the treatment of cancer. In particular, the invention relates to a method for delivering a therapeutic anti-cancer agent to a cancer cell or cancer cells or a tumor mass by optionally diagnosing a subject with a cancer, administering to the subject with cancer a composition comprising engineered ASCs which have been modified to express a gene encoding at least one anti-cancer agent, and where some cells, such as but not limited to endothelial cells within a population of the ASC administered to the subject integrate into the vascularization of the tumor cells and enhance angiogenesis, and where some cells, such as adipose-derived stem cells home into a tumor mass, thereby delivering the anti-cancer agent directly to the cancer cells through the local vascularization network of the tumor as well as from a location within close proximity to the surrounding tumor.

The method is based upon a surprising finding that, while some of the cancer therapies utilize anti-angiogenic chemicals and proteins that suppress angiogenesis around tumors, the high angiogenic activity of the tumors can be used to advance distribution of anticancer agents within the tumor. Thus, the present invention is inverse to the prevailing view that tumors be treated with anti-angiogenic agents. Here, the genetically modified ASC which express an anti-cancer agent are incorporated into the vasculatures around the tumors and surreptitiously deliver the anti-cancer protein to the tumors at the same time.

In particular, ASC are particularly useful as a anti-cancer agent delivery tool as the inventors have discovered that ASC comprise a heterogeneous population of cells which can be used to deliver an anti-cancer agent via a variety of different routes, such as for example some cells integrate into the vascularization and blood vessels which provide blood to the tumor (e.g. endothelial cells) whereas other cells within a ASC population can function to specifically migrate and home into the tumor cells (e.g. stem cells and mesenchymal cells within the ASC population). Accordingly, the present invention relates to the methods and uses of engineered ASC to continuously deliver anti-cancer agents to a cancer or tumor cell mass using the vascularization network that supplies the tumor with blood and nutrients.

One aspect of the present invention relates to ASC-based delivery systems as a method for therapeutic delivery of biologically active anti-cancer agents (such as anti-cancer protein molecules, or apoptosis-inducing molecules) to a tumor or cancer cell mass.

Accordingly, the present invention provides a method to deliver a high dose of an anti-cancer therapeutic agent locally and directly to the site of a tumor. Without wishing to be bound by theory, since expressed anti-cancer agents are delivered locally and are expected to have a short half-life in the circulation, systemic non-specific responses will be minimal. This allows one to use higher concentration of the anti-cancer agents to more effectively kill the cancer cells

One aspect of the present invention relates to a method for the treatment of cancer in a subject comprising transplantation of a population of ASC to a subject with cancer, the method comprising obtaining ASC from a subject, and genetically modifying the ASC to express an anti-cancer agent, such as, for example, an anti-cancer protein, and administering the engineered ASC which express the anti-cancer agent to the subject for the treatment of cancer. In some embodiments, the method is autologous transplantation of a population of ASC to subject with cancer, and in other embodiments the method is allogenic transplantation of a population of ASC to a subject with cancer. Accordingly, the present invention provides a method for ASC-based delivery of anti-cancer agents to a tumor in a subject. In some embodiments, a population of ASCs are administered locally to a subject with cancer, such as local delivery to the site of the tumor by any method commonly known to a skilled artisan. In other embodiments, the ASCs are systemically administered to a subject with cancer by method routinely used and known by a person of ordinary skill in the art. In some embodiments, the subject to whom a composition comprising engineered ASC which comprise a nucleic acid sequence encoding an anti-cancer agent has not previously had a tumor resection. In some embodiments, a composition comprising engineered ASC as disclosed herein is not administered to promote muscle regeneration in a subject with cancer. In some embodiments, a composition comprising engineered ASC as disclosed herein is not administered at a location or a lesion site where a tumor has been removed from the subject.

One aspect of the present invention relates to methods and compositions for the treatment of cancer in a subject, where the methods comprise administering to a subject with cancer a composition comprising a substantially pure population of Adipose-derived stromal cells (ASC) which have been modified to comprise a nucleic acid sequence operatively linked to a first promoter, where the nucleic acid encodes at least one anti-cancer agent, and the expression of the nucleic acid sequence encoding the anti-cancer agent is an effective amount to treat the cancer.

In some embodiments, a ASC is obtained from the same subject to which the composition is administered. In another embodiment, a matching donor from which the ASCs are harvested may be used and administered to a subject with cancer.

In some embodiments of all aspects of the invention, an anti-cancer agent expressed by the engineered ASC is selected from the group consisting of: nucleic acid, protein, peptide, siRNA, antisense nucleic acid, asRNA, RNAi, miRNA and variants thereof.

In some embodiments of all aspects of the invention, the composition comprising a population of adipose-derived stromal cells (ASC) comprises a heterogeneous population cells, such as for example, at least one of the following cell types; to a population of endothelial cells, a population of mesenchymal cells, a population of fibroblasts, a population of smooth muscle cells, a population of pericyes, a population of adipose-derived stem cell. In some embodiments, a population of adipose-derived stromal cells (ASC) comprises at least 2, or at least 3 or at least 4 of the following cell types; to a population of endothelial cells, a population of mesenchymal cells, a population of fibroblasts, a population of smooth muscle cells, a population of pericyes, a population of adipose-derived stem cell. In some embodiments, a population of adipose-derived stromal cells (ASC) additionally comprises other cells or a cell populations not listed above.

In some embodiments of all aspects of the invention, the composition comprises a population of adipose-derived stromal cells (ASC) which comprises a substantially pure population of adipose-derived stem cells. In some embodiments, the composition comprises a population of adipose-derived stromal cells (ASC) which has been purified from adipose tissue. In some embodiments, the composition comprises a population of adipose-derived stromal cells (ASC) which has been selected and from a heterogeneous population of cells from adipose tissue.

In some embodiments of all aspects of the invention, the composition comprising a population of adipose-derived stromal cells (ASC) is administered locally to the tumor. Administration of the ASC population directly to the tumor can be achieved, for example, using ultrasound or magnetic resonance imaging. In some embodiments, one can administer the engineered ASCs to a subject during or after the subject has had an operation or surgical resection, for example a surgical procedure designed to remove part or all of a tumor mass. In another embodiment, a subject to whom the engineered ASCs are administered has not had an operation or surgical resection, for example a surgical procedure designed to remove part or all of a tumor mass. In some embodiments, the engineered ASC are not administered to a subject at the site of a surgical resection, for example a surgical procedure designed to remove part or all of a tumor mass. In some embodiments, the administration of the engineered ASCs as disclosed herein are not administered to a subject with cancer to promote wound healing following a tumor removal, to promote muscle regeneration following tumor removal.

In some embodiments of all aspects of the invention, an anti-cancer agent expressed by an engineered ASC is a pro-apoptotic molecule or apoptosis-inducing molecule or fragment thereof. In some embodiments, an anti-cancer agent expressed by an engineered ASC is not an anti-angiogenic agent, or a hormone or an angiogenic hormone (e.g. VEGF) or any agent which promotes angiogenesis or neovascularization in a subject.

In some embodiments, the nucleic acid sequence encoding at least one anti-cancer agent also encodes a secretory sequence to secrete the anti-cancer agent out of the ASC. Secretory sequences are well known in the art and any secretory sequence is encompassed for use in the present invention.

In some embodiments of all aspects of the invention, the ASC further comprises a second nucleic acid sequence operatively linked to a second promoter, wherein the second nucleic acid encodes at least one cell death gene. For example, the ASC can comprise a “self-suicide” gene, such as pro-apoptotic agent operatively linked to an inducible promoter as a biological safety mechanism to eliminate the engineered ASC from the subject when desired, for instance when delivery of the anti-cancer agent is no longer desired due to effective treatment and/or elimination of the cancer in the subject.

In some embodiments of all aspects of the invention, the subject is a mammalian subject, for example, a human subject.

In some embodiments of all aspects of the invention, the ASC is transfected ex vivo with the first nucleic acid sequence operatively linked to a first promoter. Vectors are typically used to transfect the cells.

In some embodiments of all aspects of the invention, an anti-cancer agent expressed by the engineered ASC is a polypeptide, for example, a pro-apoptotic polypeptide or pro-apoptotic molecule such as, but not limited to TRAIL, Bcl-XL, Hsp90; TNFα; DIABLO; BAX; BID; BID; BIM; inhibitors of Bcl-2; Bad; poly ADP ribose polymerase-1 (PARP-1); Second Mitochondria-derived Activator of Caspases (SMAC); apoptosis inducing factor (AIF); Fas (Apo-1 or CD95); Fas ligand (FasL) and variants and fragments thereof.

In some embodiments of all aspects of the invention, an anti-cancer polypeptide agent is a protein which activates a pro-drug, such as, but not limited to thymidine kinase, carboxypeptidase G2, purine-deoxynucleoside phosphorylase (PNP), a fusion protein consisting of yeast cytosine deaminase and uracil phosphoribosyl transferase (FCU1), cytosine deaminase and the like.

In some embodiments of all aspects of the invention, an anti-cancer agent useful to be expressed by the engineered ASC is an inhibitor to a cancer target gene, for example, where the inhibitor to a cancer target gene is a nucleic acid inhibitor, such as an oligonucleotide, antisense, a RNAi molecule, such as but not limited to siRNA, miRNA, shRNA and the like, which gene silences a cancer target gene. In such embodiments, a cancer target gene is any gene which is expressed in a cancer cell, or cancer stem cell but not expressed in non-cancer cells or non-cancer stem cells and is for example an oncogene, such as but not limited to cancer genes selected from the group of HER2/Her-2, BRAC1 and BRAC2, Rb, p53, and variants thereof.

In some embodiments of all aspects of the invention, the method are applicable to the treatment of any cancer in a subject, preferably a mammalian subject or human subject, where the cancer is for example, but not limited to mescenchymal in origin (sarcomas); fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant leukemias; lymphocytic leukemias; plasmacytomas; leiomyosarcomas; and rhabdomyosarcoma; cancers epithelial in origin (carcinomas); squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; transitional cell carcinomas; squamous cell carcinomas; choriocarcinomas; seminomas; embryonal carcinomas; malignant teratomas; and terato carcinomas; leukemia; acute lymphocytic leukemia and acute myelocytic leukemia (myeloblastic, promyeloblastic, myelomonocytic; monocytic, and erythroleukemia); chronic leukemia; chronic myelocytic (granulocytic) leukemia; chronic lymphocytic leukemia; polycythemia vera; lymphoma; Hodgkin's disease; non-Hodgkin's disease; multiple mycloma; Waldenström's macroglobulinemia; heavy chain disease. In some embodiments, the cancer is lymphia; leukemia; sarcoma; adenomas. In some embodiments, the cancer is acute lympoblastic leukemia (ALL).

Another aspect of the present invention relates to a kit for producing an engineered adipose-derived stromal cell (ASC) as disclosed herein for the treatment of cancer, wherein the kit comprises at least one vector comprising a nucleic acid sequence operatively linked to a promoter, wherein the nucleic acid sequence encodes at least one an anti-cancer agent. The kit can also comprise instructions and culture media to transduce ASC as well as instructions and culture media to proliferate engineered ASC. In addition, the kit can further comprise devices and pharmaceutically acceptable reagents for administration of the engineered ASC to the subject.

### DETAILED DESCRIPTION OF THE INVENTION

The present invention relates to methods and compositions for delivery of an anti-cancer agent to a tumor in a subject, and in particular relates to use of genetically engineered adipose-derived stromal cells (ASC) which express an anti-cancer agent for the treatment of a subject with cancer.

In particular, ASC are particularly useful as an anti-cancer agent delivery vehicle or delivery tool as the inventors have discovered that ASC specifically migrate and integrate into the vascularization surrounding the tumor and enhances angiogenesis. Accordingly, the present invention relates to the use of engineered ASC to continuously deliver an anti-cancer agent to a cancer or tumor cell mass, where some cells within the ASC population, such as but not limited to endothelial cells, integrate into the vascularization network that supplies the tumor with blood and nutrients and other cells within the ASC population, such as but not limited to adipose-derived stem cells, migrate and home into the tumor mass.

One aspect of the present invention relates to a method for delivering a therapeutic anti-cancer agent to cancer cells by administering to the subject with cancer a composition comprising a population of engineered ASCs which have been modified to express and secrete at least one anti-cancer agent, and where the engineered ASCs integrate into the vascularization surrounding the tumor cell mass and enhance angiogenesis, thereby delivering the anti-cancer agent directly into the systemic circulation and local vascularization network which supplies blood and nutrients to the cancer cells.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims.

The term “Adipose-derived stromal cells” or “ASCs” refers to adult cells that originate from adipose tissue. Adipose-derived stromal cells is a heterologous population of cells comprising at least one or at least 2 or the following population of cells; endothelial cells, mesenchymal stem cells, fibroblasts, smooth muscle cells, pericytes and adipose-derived stem cells, as well as additional other cell types not listed. In some embodiments, adipose-derived stromal cells refers to a substantially pure population of adipose-derived stem cells. In some embodiments, adipose-derived stromal cells does not refers to adipose derived regenerative cells. ASC are harvested from adipose tissue and are substantially free of adipocytes and red blood cells and clonal populations of connective tissue stem cells. The ASCs are substantially devoid of cells, which includes extracellular matrix material from adipose tissue.

The term “adipose” as used herein refers to any fat tissue from a subject. The terms “adipose” and “adipose tissue” are used interchangeably herein. The adipose tissue may be brown fat, white fat or yellow fat or white adipose tissue, derived from subcutaneous, omental/visceral, mammary, gonadal, or other adipose tissue site. The adipose tissue has adipocytes and stroma. Adipose tissue is found throughout the body of an animal. For example, in mammals, adipose tissue is present in the omentum, bone marrow, subcutaneous space and surrounding most organs. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably, the adipose tissue is human. A convenient source of adipose tissue is from liposuction surgery, however, the source of adipose tissue or the method of isolation of adipose tissue is not critical to the invention.

The term “adult” as used herein, is meant to refer to any non-embryonic or non-postnatal juvenile animal or subject. For example the term “adult adipose-derived stromal cell,” refers to an adipose-derived stromal cell, other than that obtained from an embryo or juvenile animal.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” or “biologically active” molecule is a molecule in a form in which it exhibits a property or activity by which it is characterized.

The term “graft” as used herein refers to the process whereby a free (unattached) cell, tissue, or organ integrates into a tissue following transplantation into a subject.

The term “allograft” refers to a transplanted cell, tissue, or organ derived from a different animal of the same species.

The term “xenograft” as used herein refers to a transplanted cell, tissue, or organ derived from an animal of a different species.

The term “homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology. As used herein, “homology” is used synonymously with “identity.”

The term “isolated,” when used in reference to cells, refers to a single cell of interest, or a heterogeneous population of cells of interest such as adipose-derived stromal cells, at least partially isolated from other cell types or other cellular material with which it naturally occurs in the tissue of origin (e.g., adipose tissue). Stated another way, isolated ASC are substantially free of adipocytes and red blood cells and clonal populations of connective tissue stem cells, and are substantially devoid of cells such as extracellular matrix material and cells from adipose tissue. A sample of adipose-derived stromal cells which is “substantially pure” when it is at least 60%, or at least 75%, or at least 90%, and, in certain cases, at least 99% free of cells of adipose tissue other than cells of interest. For clarity, the cells of interest in a heterogeneous population of cells of an ASC population include, for example but are not limited to endothelial cells, mesenchymal stem cells, fibroblasts, smooth muscle cells, pericytes and adipose-derived stem cells. Purity can be measured by any appropriate method, for example, by fluorescence-activated cell sorting (FACS), or other assays which distinguish cell types.

As used herein, the term “purified”, relates to an enrichment of a cell, cell type, molecule, or compound relative to other components normally associated with the cell, cell type, molecule, or compound in a native environment. The term “purified” does not necessarily indicate that complete purity of the particular cell, cell type, molecule, or compound has been achieved during the process. A “highly purified” population of ASC as used herein refers to a population of ASC that is greater than 90% pure (i.e. the highly purified population of ASC comprises at least 90% cells of ASC population (i.e. endothelial cells, mesenchymal stem cells, fibroblasts, smooth muscle cells, pericytes and adipose-derived stem cells) relative to non-ASC cells such as red blood cells, adipocytes and cells of the extracellular matrix of adipose tissue).

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, a “subject” is any organism or animal to whom which treatment or prophylaxis treatment is desired. Such animals include mammals, preferably a human.

The term “gene” as used herein refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript, which are termed “5′ untranslated regions” or 5′UTR and 3′ untranslated regions (3′UTR) respectively. These sequences are also referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

The term “exon” as used herein refers to the normal sense of the term as meaning a segment of nucleic acid molecules, usually DNA, that encodes part of or all of an expressed protein.

The term “expression” as used herein refers to the biosynthesis of a gene product, preferably to the transcription and/or translation of a nucleotide sequence, for example an endogenous gene or a heterologous gene, in a cell. For example, in the case of a heterologous nucleic acid sequence, expression involves transcription of the heterologous nucleic acid sequence into mRNA and, optionally, the subsequent translation of mRNA into one or more polypeptides. Expression also refers to biosynthesis of a RNAi molecule, which refers to expression and transcription of an RNAi agent such as siRNA, shRNA, and antisense DNA but does not require translation to polypeptide sequences.

The term “expression construct” and “nucleic acid construct” as used herein are synonyms and refer to a nucleic acid sequence capable of directing the expression of a particular nucleotide sequence, such as the heterologous target gene sequence in an appropriate host cell (e.g., a mammalian cell). If translation of the desired heterologous target gene is required, it also typically comprises sequences required for proper translation of the nucleotide sequence. The coding region may code for a protein of interest but may also code for a functional RNA of interest, for example antisense RNA, dsRNA, or a nontranslated RNA, in the sense or antisense direction. The nucleic acid construct as disclosed herein can be chimeric, meaning that at least one of its components is heterologous with respect to at least one of its other components.

The term “gene” as used herein refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., 3′UTR, 5″UTR, introns) between individual coding regions (i.e., exons). The term “structural gene” as used herein is intended to mean a DNA sequence that is transcribed into mRNA which is then translated into a sequence of amino acids characteristic of a specific polypeptide.

The term “gene product(s)” as used herein refers to include RNA transcribed from a gene, or a polypeptide encoded by a gene or translated from RNA.

The terms “genome” or “genomic DNA” as used herein refers to the heritable genetic information of a host organism. Genomic DNA comprises the DNA of the nucleus (also referred to as chromosomal DNA) but also the DNA of the plastids (e.g., chloroplasts) and other cellular organelles (e.g., mitochondria). The terms genome or genomic DNA typically refers to the chromosomal DNA of the nucleus.

The terms “heterologous target gene” or “heterologous gene sequence” are used interchangeably herein refers to any nucleic acid (e.g., gene sequence) which is introduced into the genome of a cell. Heterologous gene sequences may include gene sequences found in that cell so long as the introduced gene to be expressed at different levels as compared to the level naturally occurring in the host cell and/or contains some modification (e.g., a point mutation, the presence of a selectable marker gene, etc.) relative to the naturally-occurring gene, or is not expressed at the same level normally in the cells as compared to the level which is being induced. A heterologous target gene can be present in the cell but not at the levels being expressed, or the nucleic acid sequence has been modified by experimental manipulations, such as example, modification being a substitution, addition, deletion, inversion or insertion of one or more nucleotide residues, for example modified by non-natural, synthetic or “artificial” methods such as, for example mutagenesis or the nucleic acid is not located in its natural or native genetic environment. Such methods for nucleic acid modification have been described (U.S. Pat. No. 5,565,350; WO 00/15815 which is incorporated herein by reference). In some instances a heterologous target gene includes, but are not limited to, coding sequences of heterologous genes or structural genes (e.g., reporter genes, selection marker genes, drug resistance genes, growth factors, etc.), and non-coding regulatory sequences which do not encode an mRNA or protein product, (e.g., promoter sequence, polyadenylation sequence, termination sequence, enhancer sequence, RNAi molecules etc.). A nucleic acid sequence of interest may preferably encode for a heterologous gene, for example a valuable trait, for example but not limited to, a heterologous gene encoding a toxin protein, or fragment thereof for use in cancer therapeutics etc. In some instances, a heterologous target gene is not endogenous to or not naturally associated with the cell into which it is introduced, but has been obtained from another cell. Heterologous target genes also includes an endogenous DNA sequence, which contains some modification, non-naturally occurring multiple copies of a endogenous DNA sequence, or a DNA sequence which is not naturally associated with another DNA sequence physically linked thereto. Generally, although not necessarily, heterologous target genes encodes RNA and proteins that are not normally produced by the cell into which it is expressed.

The terms “target”, “target gene” and “target nucleotide sequence” are used equivalently herein and refers to a target gene can be any gene of interest present in an organism. A target gene may be endogenous or introduced. For example, a target gene is a gene of known function or is a gene whose function is unknown, but whose total or partial nucleotide sequence is known. Alternatively, the function of a target gene and its nucleotide sequence are both unknown. A target gene can be a native gene of the eukaryotic cell or can be a heterologous gene which has previously been introduced into the eukaryotic cell or a parent cell of said eukaryotic cell, for example by genetic transformation. A heterologous target gene can be stably integrated in the genome of the eukaryotic cell or is present in the eukaryotic cell as an extrachromosomal molecule, e.g. as an autonomously replicating extrachromosomal molecule. A target gene can include polynucleotides comprising a region that encodes a polypeptide or polynucleotide region that regulates replication, transcription, translation, or other process important in expression of the target protein; or a polynucleotide comprising a region that encodes the target polypeptide and a region that regulates expression of the target polypeptide; or non-coding regions such as the 5′ or 3′ UTR or introns. A target gene may refer to, for example, an mRNA molecule produced by transcription a gene of interest.

The term “endogenous” nucleotide sequence refers to a nucleotide sequence, which is present in the genome of the cell under normal conditions, i.e. a nucleotide sequence which present normally in the cell and is not introduced into the cell or by other genetic manipulation strategies. A nucleic acid sequence referred to as a “non-endogenous” or “synthetic” sequence refers to a sequence, where the entire sequence is not found in the cell to which the nucleic acid is introduced. In some embodiments, the RNAi target site is a non-endogenous or synthetic sequence, meaning the entire sequence is not found within the cell that the nucleic acid construct is introduced into.

The term an “essential” gene is a gene encoding a protein such as e.g. a biosynthetic enzyme, receptor, signal transduction protein, structural gene product, or transport protein that is essential to the growth or survival of the organism or cell.

The term “homologous DNA Sequence” as used herein refers to a DNA sequence naturally associated with a host cell or another DNA sequence.

The term “mammal” or “mammalian” are used interchangeably herein, are intended to encompass their normal meaning. While the invention is most desirably intended for efficacy in humans, it may also be employed in domestic mammals such as canines, felines, and equines, as well as in mammals of particular interest, e.g., zoo animals, farmstock, transgenic animals, rodents and the like.

As used herein, “gene silencing” or “gene silenced” in reference to an activity of a RNAi molecule, for example a siRNA or miRNA refers to a decrease in the mRNA level in a cell for a heterologous target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. As used herein, the “reduced” or “gene silencing” refers to lower, preferably significantly lower, more preferably the expression of the nucleotide sequence is not detectable.

The term “double-stranded RNA” molecule, “RNAi molecule”, or “dsRNA” molecule as used herein refers to a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. In some embodiments, the terms refer to a double-stranded RNA molecule capable, when expressed, is at least partially reducing the level of the mRNA of the heterologous target gene. In particular, the RNAi molecule is complementary to a synthetic RNAi target sequence located in a non-coding region of the heterologous target gene. As used herein, “RNA interference”, “RNAi”, and “dsRNAi” are used interchangeably herein refer to nucleic acid molecules capable of gene silencing.

As used herein, the term “RNAi” refers to any type of interfering RNA, including but are not limited to, siRNAi, shRNAi, stRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length).

As used herein “shRNA” or “small hairpin RNA” (also called stem loop) is a type of siRNA. In one embodiment, these shRNAs are composed of a short, e.g. about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches. In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

As used herein the term “hairpin RNA” refers to any self-annealing double stranded RNA molecule. In its simplest representation, a hairpin RNA consists of a double stranded stem made up by the annealing RNA strands, connected by a single stranded RNA loop, and is also referred to as a “pan-handle RNA”. However, the term “hairpin RNA” is also intended to encompass more complicated secondary RNA structures comprising self-annealing double stranded RNA sequences, but also internal bulges and loops. The specific secondary structure adapted will be determined by the free energy of the RNA molecule, and can be predicted for different situations using appropriate software such as FOLDRNA (Zuker and Stiegler (1981) Nucleic Acids Res 9(1):133-48; Zuker, M. (1989) Methods Enzymol. 180, 262-288).

The term “agent” refers to any entity which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “a reduction” of the level of a gene, included a decrease in the level of a protein or mRNA means in the cell or organism. As used herein, “at least a partial reduction” of the level of an agent (such as a RNA, mRNA, rRNA, tRNA expressed by the target gene and/or of the protein product encoded by it) means that the level is reduced at least 25%, preferably at least 50%, relative to a cell or organism lacking the RNAi agent as disclosed herein. As used herein, “a substantial reduction” of the level of an agent such as a protein or mRNA means that the level is reduced relative to a cell or organism lacking a chimeric RNA molecule of the invention capable of reducing the agent, where the reduction of the level of the agent is at least 75%, preferably at least 85%. The reduction can be determined by methods with which the skilled worker is familiar. Thus, the reduction of the transgene protein can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) to detect transgene protein or mRNA. Depending on the type of the reduced transgene, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “antisense” as used herein refers to a nucleotide sequence that is inverted relative to its normal orientation for transcription and so it can hybridize to the target gene mRNA molecule through Watson-Crick base pairing. An antisense strand may be constructed in a number of different ways, provided that it is capable of interfering with the expression of a target gene. For example, the antisense strand can be constructed by inverting the coding region (or a portion thereof) of the target gene relative to its normal orientation for transcription to allow the transcription of its complement, (e.g., RNAs encoded by the antisense and sense gene may be complementary). Furthermore, the antisense oligonucleotide strand need not have the same intron or exon pattern as the target gene, and noncoding segments of the target gene may be equally effective in achieving antisense suppression of target gene expression as coding segments. In the context of gene silencing the term “antisense” is understood to mean a nucleic acid having a sequence complementary to a target sequence, for example a messenger RNA (mRNA) sequence the blocking of whose expression is sought to be initiated by hybridization with the target sequence.

The term “mature protein” as used herein refers to a protein which is normally synthesized in pre-form, such as, for example, a pre-protein comprising a protein plus additional protein components, such as targeting or signal peptides, which direct translocation to a specific cellular organelle. Post-translation processing of the pre-protein results in the mature protein, for example cleavage of the pre-protein components. In some instances, a pre-protein can comprise a biologically inactive protein, which on post-translation processing renders an active protein.

The term “minimal promoter” as used herein refers to the minimal nucleic acid regions of promoter elements while also maintaining a functional promoter.

The term “non-coding” refers to sequences of nucleic acid molecules that do not encode part or all of an expressed protein. Non-coding sequences include but are not limited to introns, promoter regions, 3′ untranslated regions (3′UTR), and 5′ untranslated regions (5′UTR).

The terms “nucleic acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. shRNAs also can comprise non-natural elements such as non-natural bases, e.g., ionosin and xanthine, nonnatural sugars, e.g., 2′-methoxy ribose, or non-natural phosphodiester linkages, e.g., methylphosphonates, phosphorothioates and peptides. The term “nucleic acid” or “oligonucleotide” or “polynucleotide” are used interchangeably herein and refers to at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

The term “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O- and N-alkylated nucleotides, e.g. N6-methyl adenosine are suitable. The 2′OH-group can be replaced by a group selected from H. OR, R. halo, SH, SR, NH₂, NHR, NR₂ or CN, wherein R is C-C6 alkyl, alkenyl or alkynyl and halo is F. C1, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The term “oligonucleotide” as used herein refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

The term “operable linkage” or “operably linked” are used interchangeably herein, are to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of the linked nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. In some embodiments, arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly can be any distance, and in some embodiments is less than 200 base pairs, especially less than 100 base pairs, less than 50 base pairs. In some embodiments, the nucleic acid sequence to be transcribed is located behind the promoter in such a way that the transcription start is identical with the desired beginning of the chimeric RNA of the invention. Operable linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. In some embodiments, the expression construct, consisting of a linkage of promoter and nucleic acid sequence to be expressed, can exist in a vector integrated form and be inserted into a plant genome, for example by transformation.

The term “nucleic acid construct” as used herein refers to a nucleic acid at least partly created by recombinant methods. The term “DNA construct” is referring to a polynucleotide construct consisting of deoxyribonucleotides. The construct can be single or double stranded. The construct can be circular or linear. A person of ordinary skill in the art is familiar with a variety of ways to obtain one of a DNA construct. Constructs can be prepared by means of customary recombination and cloning techniques as are described, for example, in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc and Wiley Interscience; Gelvin et al. (Eds) (1990) Plant Molecular Biology Manual; Kluwer Academic Publisher, Dordrecht, The Netherlands.

The terms “polypeptide”, “peptide”, “oligopeptide”, “polypeptide”, “gene product”, “expression product” and “protein” are used interchangeably herein to refer to a polymer or oligomer of consecutive amino acid residues.

The terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur (e.g., tissue promoters). If a promoter is an inducible promoter, then the rate of transcription in creases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as in a cancer cell.

The term “tissue specific” as it applies to a promoter refers to a promoter that is capable of directing selective expression of a nucleotide sequence of interest to a specific type of tissue (e.g., liver) in the relative absence of expression of the same nucleotide sequence of interest in a different type of tissue (e.g., kidney). Tissue specificity of a promoter may be evaluated by, for example, operably linking a reporter gene to the promoter sequence to generate a reporter construct, introducing the reporter construct into the genome of an organism, e.g. an animal model such that the reporter construct is integrated into every tissue of the resulting transgenic animal, and detecting the expression of the reporter gene (e.g., detecting mRNA, protein, or the activity of a protein encoded by the reporter gene) in different tissues of the transgenic animal. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining or immunohistochemical staining.

The term “constitutive” when made in reference to a promoter means that the promoter is capable of directing transcription of an operably linked nucleic acid sequence in the absence of a stimulus (e.g., heat shock, chemicals, agents, light, etc.). Typically, constitutive promoters are capable of directing expression of a transgene in substantially any cell and any tissue.

In contrast, the term “regulatable” or “inducible” promoter referred to herein is one which is capable of directing a level of transcription of an operably linked nuclei acid sequence in the presence of a stimulus (e.g., heat shock, chemicals, light, agent etc.) which is different from the level of transcription of the operably linked nucleic acid sequence in the absence of the stimulus.

The term “sense” as used herein refers to a nucleic acid having a sequence which is homologous or identical to a target sequence, for example a sequence which binds to a protein transcription factor and which is involved in the expression of a given gene. According to a preferred embodiment, the nucleic acid comprises a gene of interest and elements allowing the expression of the said gene of interest.

In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

In its broadest sense, the term “substantially identical”, when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of 10-22 nucleotides, such as at least 10, 11, 12, 13, 14, 15, 16, 17, 18, 19, 20, 21, 22 or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially identical” to a reference nucleotide sequence hybridizes to the exact complementary sequence of the reference nucleotide sequence (i.e. its corresponding strand in a double-stranded molecule) under low stringency conditions, preferably medium stringency conditions, most preferably high stringency conditions (as defined above). Homologues of a specific nucleotide sequence include nucleotide sequences that encode an amino acid sequence that is at least 24% identical, at least 35% identical, at least 50% identical, at least 65% identical to the reference amino acid sequence, as measured using the parameters described above, wherein the amino acid sequence encoded by the homolog has the same biological activity as the protein encoded by the specific nucleotide. The term “substantially non-identical” refers to a nucleotide sequence that does not hybridize to the nucleic acid sequence under stringent conditions. The term “substantially identical”, when used herein with respect to a polypeptide, means a protein corresponding to a reference polypeptide, wherein the polypeptide has substantially the same structure and function as the reference protein, e.g. where only changes in amino acids sequence not affecting the polypeptide function occur. When used for a polypeptide or an amino acid sequence, the percentage of identity between the substantially similar and the reference polypeptide or amino acid sequence is at least 24%, at least 30%, at least 45%, at least 60%, at least 75%, at least 90%, at least 95%, at least 99%, using default GAP analysis parameters as described above. Homologues are amino acid sequences that are at least 24% identical, more preferably at least 35% identical, yet more preferably at least 50% identical, yet more preferably at least 65% identical to the reference polypeptide or amino acid sequence, as measured using the parameters described above, wherein the amino acid sequence encoded by the homolog has the same biological activity as the reference polypeptide.

The term “transformation” as used herein refers to the introduction of genetic material (e.g., a transgene or heterologous nucleic acid molecules) into a cell, tissue or organism. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme linked immunosorbent assay (ELISA), which detects the presence of a polypeptide encoded by one or more of the transgenes. Alternatively, transient transformation may be detected by detecting the activity of the protein (e.g., β-glucuronidase) encoded by the transgene (e.g., the uid A gene). The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences, which are capable of binding to one or more of the transgenes. Alternatively, stable transformation of a cell may also be detected by the polymerase chain reaction of genomic DNA of the cell to amplify transgene sequences. The term “stable transformant” refers to a cell, which has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression, which may exhibit variable properties with respect to meiotic stability. Transformed cells, tissues, or plants are understood to encompass not only the end product of a transformation process, but also transgenic progeny thereof.

The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence. The term transgenic when referring to a cell, tissue or organisms means transformed, preferably stably transformed, with a recombinant DNA molecule that preferably comprises a suitable promoter operatively linked to a DNA sequence of interest.

The term “vectors” is used interchangeably with “plasmid” to refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences under the control of any RNA polymerase, including mitochondrial RNA polymerase, RNA pol I, RNA pol II, and RNA pol III. Vectors may be desirably designed to utilize an endogenous mitochondrial RNA polymerase (e.g., human mitochondrial RNA polymerase, in which case such vectors may utilize the corresponding human mitochondrial promoter). Mitochondrial polymerases may be used to generate capped (through expression of a capping enzyme) or uncapped messages in vivo. RNA pol I, RNA pol II, and RNA pol III transcripts may also be generated in vivo. Such RNAs may be capped or not, and if desired, cytoplasmic capping may be accomplished by various means including use of a capping enzyme such as a vaccinia capping enzyme or an alpha virus capping enzyme. Such plasmids or vectors can include plasmid sequences from bacteria, viruses or phages. Such vectors include chromosomal, episomal and virus-derived vectors e.g., vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. Thus, one exemplary vector is a single or double-stranded phage vector. Another exemplary vector is a single or double-stranded RNA or DNA viral vector. Such vectors may be introduced into cells as polynucleotides, preferably DNA, by well known techniques for introducing DNA and RNA into cells. The vectors, in the case of phage and viral vectors may also be and preferably are introduced into cells as packaged or encapsidated virus by well known techniques for infection and transduction. Viral vectors may be replication competent or replication defective. In the latter case, viral propagation generally occurs only in complementing host cells.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection.

The terms “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. The term “tumor” or “tumor cell” are used interchangeably herein, refers to the tissue mass or tissue type of cell that is undergoing abnormal proliferation.

As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

In this specification and the appended claims, the singular forms “a,” “an,” and “the” include plural references unless the context clearly dictates otherwise, and therefore “a” and “an” are used herein to refer to one or to more than one (i.e., at least one) of the grammatical object of the article. By way of example, “an element” means one element or more than one element, and reference to a composition for delivering “an agent” includes reference to one or more agents.

Compositions or methods “comprising” one or more recited elements may include other elements not specifically recited. For example, a composition that comprises ASC encompasses both the isolated ASC but may also include other cell types or protein or other components. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

Other than in the operating examples, or where otherwise indicated, all numbers expressing quantities of ingredients or reaction conditions used herein should be understood as modified in all instances by the term “about.” The term “about” is used herein to mean approximately, roughly, around, or in the region of. When the term “about” is used in conjunction with a numerical range, it modifies that range by extending the boundaries above and below the numerical values set forth. The term “about” when used in connection with percentages will mean ±1%.

### Adipose-Derived Stromal Cells

Without wishing to be bound to theory, adipose tissue plays an important and overlooked role in the normal development and physiology of humans and other mammalian species. Many different kinds of fat exist. The most common type is white adipose tissue, located under the skin (subcutaneous fat), within the abdominal cavity (visceral fat) and around the reproductive organs (gonadal fat). Less common in the adult human is brown adipose tissue, which plays an important role in generating heat during the neonatal period; this type of fat is located between the shoulder blades (interscapular), around the major vessels and heart (periaortic and pericardial), and above the kidney (suprarenal). Adipose tissue also encompasses yellow fat. Adipose tissue is found throughout the body of an animal, including humans, and is present in the omementum, bone marrow, subcutaneous space and surrounding most organs.

Adult ASC or human adipose tissue-derived adult stromal cells represent a cell source that can be harvested routinely with minimal risk or discomfort to the subject. They can be expanded ex vivo, differentiated along unique lineage pathways, genetically engineered, and re-introduced into individuals as either autologous or allogenic transplantation.

A population of ASCs as described herein, comprise is a heterologous population of cells comprising at least one or at least 2 or the following population of cells; endothelial cells, mesenchymal stem cells, fibroblasts, smooth muscle cells, pericytes and adipose-derived stem cells, as well as additional other cell types not listed. In some embodiments, adipose-derived stromal cells refers to a substantially pure population of adipose-derived stem cells. In some embodiments, adipose-derived stromal cells does not refers to adipose derived regenerative cells. ASC useful in the methods of the present invention have the ability to differentiate into various cell types, including, but no limited to, adipocytes, chondrocytes, and osteoblasts, as well as provide fully differentiated and functional cells for research, transplantation, and development of tissue engineering products for the treatment of diseases and disorders and traumatic injury repair.

ASCs can be cultured according to method commonly known in the art to induce the ASCs to give rise to cells having a mesodermal, ectodermal or endodermal lineage. After culturing ASCs in the differentiating-inducing medium for a suitable time (e.g., several days to a week or more), the ASCs can be assayed to determine whether, in fact, they have acquired the desired lineage.

Methods to characterize a population of ASCs, include, but are not limited to, histological, morphological, biochemical and immunohistochemical methods, or using cell surface markers, or genetically or molecularly, or by identifying factors secreted by the differentiated cell, and by the inductive qualities of the differentiated ASCs.

For example, molecular markers that characterize mesodermal cell that differentiate from the ASCs of the invention, include, but are not limited to, MyoDu, myosin, alpha-actin, brachyury, FOG, tbx5 FoxF1, Nkx-2.5. Mammalian homologs of the above mentioned markers are preferred.

Molecular markers that characterize ectodermal cell that differentiate from the ASCs of the invention, include for example, but are not limited to N-CAM, GABA and epidermis specific keratin. Mammalian homologs of the above mentioned markers are preferred. Molecular markers that characterize endodermal cells that differentiate from the ADSCs include for example, but are not limited to, Xhbox8, Endo1, Xhex, Xcad2, Edd, EF1-alpha, HNF3-beta, LFABP, albumin, insulin. Mammalian homologs of the above mentioned markers are preferred.

Adipose tissue is readily accessible and abundant in many individuals. Obesity is a condition of epidemic proportions in the United States, where over 50% of adults exceed the recommended BMI based on their height.

Adult adipose-derived stromal cells (ASC) can be harvested from a subject, for example, using the methods and devices as disclosed in U.S. Pat. No. 7,270,996, which is incorporated herein by reference. Additionally, adult adipose-derived stromal cells (ASC) can be obtained and cultured according to the culture conditions as disclosed in U.S. Patent Application 2008/0248003 which is incorporated herein by reference.

ASCs useful in the methods of invention can be isolated by a variety of methods known to those skilled in the art such as described in US Patent Application 2003/0082152 or WO 00/53795, which are incorporated herein in their entirety by reference. In some embodiments, adipose tissue is isolated from a mammalian subject, preferably a human subject. In some embodiments, a source of adipose is subcutaneous adipose tissue. In some embodiments, a source of adipose tissue is omental adipose. In humans, the adipose is typically isolated by liposuction. In some embodiments, where engineered ASC are to be transplanted into a human subject, it is preferable that the adipose tissue be isolated from that same subject to provide for an autologous transplant. Alternatively, the transplanted ASCs are allogeneic.

Methods for the isolation, expansion, and differentiation of human adipose-derived stromal cells (ASC) have been reported. See for example, Burris et al. 1999, Mol Endocrinol 13:410-7; Erickson et al. 2002, Biochem Biophys Res Commun. Jan. 18, 2002; 290(2):763-9; Gronthos et al. 2001, Journal of Cellular Physiology, 189:54-63; Halvorsen et al. 2001, Metabolism 50:407-413; Halvorsen et al. 2001, Tissue Eng. 7(6):729-41; Harp et al. 2001, Biochem Biophys Res Commun 281:907-912; Saladin et al. 1999, Cell Growth & Diff 10:43-48; Sen et al. 2001, Journal of Cellular Biochemistry 81:312-319; Zhou et al. 1999, Biotechnol. Techniques 13: 513-517. Adipose tissue-derived stromal cells are obtained from minced human adipose tissue by collagenase digestion and differential centrifugation [Halvorsen et al. 2001, Metabolism 50:407-413; Hauner et al. 1989, J Clin Invest 84:1663-1670; Rodbell et al. 1966, J Biol Chem 241:130-139].

It is well documented that adipocytes are a replenishable cell population. Even after surgical removal by liposuction or other procedures, it is common to see a recurrence of adipocytes in an individual over time. This suggests that adipose tissue contains stromal stem cells and/or precursors that are capable of self-renewal.

However obtained, the adipose tissue is processed to separate the ASCs of the invention from the remainder of the adipose tissue. The ASC population that contains a heterogeneous population of mesenchymal stem cells, fibroblasts, smooth muscle cells and pericytes and adipose-derived stem cells is obtained by washing the obtained adipose tissue with a physiologically-compatible solution, such as phosphate buffer saline (PBS). The washing step consists of rinsing the adipose tissue with PBS, agitating the tissue, and allowing the tissue to settle. In addition to washing, the adipose tissue is dissociated. The dissociation can occur by enzyme degradation and neutralization. Alternatively, or in conjunction with such enzymatic treatment, other dissociation methods can be used such as mechanical agitation, sonic energy, or thermal energy. Three layers form after the washing, dissociation, and settling steps. The top layer is a free lipid layer. The middle layer includes the lattice and adipocyte aggregates. The middle layer is referred to as an “adipose-derived lattice enriched fraction.”

The bottom layer contains the ASC population. The bottom layer is further processed to isolate the ASCs as disclosed herein. The cellular fraction of the bottom layer is concentrated into a pellet. One method to concentrate the cells includes centrifugation.

The bottom layer is centrifuged and the pellet is retained. The pellet is designated the adipose-derived stromal cell population which includes the adipose-derived stem cells as well as other cells in the ASC population. The ASC population can also contain erythrocytes (RBCs). In a preferred method the RBCs are lysed and removed. Methods for lysis and removed RBCs are well known in the art (e.g., incubation in hypotonic medium). However, the RBCs are not required to be removed from the ADSC-EF.

The pellet is resuspended and can be washed (in PBS), centrifuged, and resuspended one or more successive times to achieve greater purity of the ASCs. The ASC population as disclosed herein is a heterogenous population of cells which include, among other cells, adipose-derived stem cells (ADSCs). The cells of the washed and resuspended pellet are ready for genetic manipulation and subsequent transplantation into a subject.

The ASCs in the resuspended pellet can be separated from other cells of the resuspended pellet by methods that include, but are not limited to, cell sorting, size fractionation, granularity, density, molecularly, morphologically, and immunohistologically. The immunophenotype of the adipose-derived stromal cells based on flow cytometry include Stromal cell-associated markers, such as CD13, CD29, CD34, CD44, CD63, CD73, CD90, CD166, as well as aldehyde dehydrogenase and the multidrug-resistance transport protein (ABCG2). ASC can also express endothelial cell-associated markers, such as for example but not limited to, CD31, CD144 or VE-cadherin, vascular endothelial growth factor receptor 2, von Willebrand factor.

In one embodiment, the ASCs are separated from the other cells on the basis of cell size and granularity where ASCs are small and agranular. Alternatively, a molecular method for separating the ASCs from the other cells of the pellet is by assaying the length of the telomere. Adipose-derived stem cells (ADSCs) tend to have longer telomeres than differentiated cells.

In another embodiment, a biochemical method for separating the ASCs from the other cells of the pellet is used by assaying telomerase activity. Telomerase activity can serve as a stem cell-specific marker.

In still another embodiment, the ASCs are separated from the other cells of the pellet immunohistochemically, for example, by panning, using magnetic beads, or affinity chromatography.

Alternatively, the process of isolating the ADSC enriched fraction with the ASCs is with a suitable device, many of which are known in the art (see, e.g., U.S. Pat. No. 5,786,207). Such devices can mechanically achieve the washing and dissociation steps.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there about at least 8.6×10⁴ or at least 8.6×10⁶ stromal cells per gram of adipose tissue (Sen et al., 2001, J. Cell. Biochem., 81:312-319). Ex vivo expansion over 2 to 4 weeks yields up to 500 million stromal cells from 0.5 kilograms of adipose tissue.

Accordingly, in some embodiments engineered ASC as disclosed herein can be used immediately for transplantation or administration to a subject with cancer or cryopreserved for future autologous or allogeneic applications to subjects with cancer.

ASC's also express a number of adhesion and surface proteins. These include cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

ASC useful to be engineered to express an anti-cancer agents according to the methods as disclosed herein can be isolated by a variety of methods known to those skilled in the art, such as described in WO 00/5379. In one embodiment, adipose tissue is isolated from a mammalian subject, preferably a human subject. In humans, the adipose is typically isolated by liposuction. If the cells of the invention are to be transplanted into a human subject, it is preferable that the adipose tissue be isolated from that same subject to provide for an autologous transplant. Alternatively, the transplanted cells are allogeneic.

Cells described herein can be isolated from adipose tissue using methods previously described (Zuk et al., Tissue Engineering 7:211, 2001; Katz et al., Stem Cells 23:412, 2005). However, one of ordinary skill in the art will appreciate that culture conditions such as cell seeding densities can be selected for each experimental condition or intended use. Other techniques useful for isolating and characterizing the cells described herein include fractionating cells using cell markers.

US 2002/0076400 and WO 00/53795 (which are incorporated herein by reference) describe the production of multipotent cell populations from human adipose tissue. Said cell populations can be differentiated into adipocytes, osteoblasts, chondrocytes, and myocytes. The publications indicate that some of the cells they can be maintained in culture in vitro for at least 15 cell transfers without losing their multipotent character. U.S. Pat. No. 6,800,480, which is incorporated herein by reference, describes methods and materials for growing primate-derived primordial stem cells in a feeder cell-free culture system.

Many techniques are known to those of ordinary skill in the art for measuring adipocyte differentiation, as well as the differentiation of other mesenchymal cells and those not described herein are encompassed within the techniques of the invention.

In one embodiment, adipose tissue, or ASC derived from adipose tissue, are subjected to varied culture media conditions as described herein to support growth or differentiation under serum-free or low serum conditions. One of ordinary skill in the art will appreciate that the amount of each growth factor, hormone, compound, nutrient, vitamin, etc., used may vary according to the culture conditions, amount of additional differentiation-inducing agent used, or the number of combination of agents used when more than one agent is used.

### Anti-Cancer Agents.

As disclosed herein, the present invention relates to engineering an adipose-derived stromal cell (ASC) to express an anti-cancer agent. An anti-cancer agent can be any agent as that term is defined herein. In some embodiments, an anti-cancer agent specifically excludes anti-angiogenic agents or hormones, such as pro-angiogenic hormones which promote angiogenesis (blood vessel growth and endothelial cell migration) and neovascularization.

In some embodiments, an anti-cancer agent is a protein, polypeptide or peptide. In some embodiments, where an anti-cancer agent is a protein, an anti-cancer agents is an pro-apoptotic protein. Pro-apoptotic proteins are well known in the art, and are encompassed for use in the methods and compositions as disclosed herein. Examples of pro-apoptotic proteins include, but are not limited to, any molecule capable of inducing a cell death pathway in a cell. Examples of such effector molecules include, but are not limited to, pro-apoptotic molecule which are well known in the art, for example but not limited to TRAIL, Hsp90; TNFα; DIABLO; BAX; BID; BID; BIM; inhibitors of Bcl-2; Bad; poly ADP ribose polymerase-1 (PARP-1); Second Mitochondria-derived Activator of Caspases (SMAC); apoptosis inducing factor (AIF); Fas (also known as Apo-1 or CD95); Fas ligand (FasL) are encompassed for use as an pro-apoptotic agents expressed by the ASC by the methods as disclosed herein, as well as natural variants or recombinant or genetically modified variants of such pro-apoptotic molecules, including biologically active truncated variants thereof.

In some embodiments, an anti-cancer agent is protein or polypeptide anti-cancer agent which selectively targets, and preferably eliminates a cancer stem cell. Such anti-cancer agents are known in the art, and include anti-cancer-agents which comprise cell-surface targeting moieties which target markers expressed on the surface of cancer stem cells.

Angiogenesis plays a central role in the pathogenesis of malignant disease and tumor growth, invasion, and metastasis. As the present invention relies on ASC to stimulate angiogenesis for delivery of the anti-cancer agent to a tumor, in one embodiment the anti-cancer encodes a therapeutic polypeptide which as an ability to induce an immune response and/or an anti-angiogenic response in vivo.

In one embodiment, a nucleic acid sequence expressed by the engineered ASC encodes a therapeutic gene that displays both immunostimulatory and anti-angiogenic activities, for example, IL12 (see Dias et al. (1998) lnt J Cancer 75(1): 151-157, and references cited herein below), interferon-alpha (INFα) (O'Byrne et al. (2000) Eur J Cancer 36(2): 151-169, and references cited therein), or a chemokine (Nomura & Hasegawa (2000) Anticancer Res 20(6A):4073-4080, and references cited therein).

In some embodiments, an anti-cancer agent expressed by the ASC can be any molecule which promotes killing of a cell, for example but not limited to, cre recombinase (Cre), pro-apoptotic genes, cytotoxin molecules, immunotoxin molecules and fragment or derivative thereof. In some embodiments, a cytotoxic molecule can be, for example but not limited to plant halotoxins, plant hemitoxins, bacterial toxins, antrax toxins, diptherial toxins (DT), pseudomonal endotoxins, sterptolysin O, saporin (SAP), pokeweek antiviral protein (PAP), bryodin 1, bouganin and gelonin and fragments or variants thereof. In some embodiments, is a molecule that sensitizes the cell to one or more secondary agents, for example an anti-cancer agent that sensitizes the cell to one or more secondary agents such as, for example; β-glutonidase, hypoxanethine-guiane phosphoribosynitransferas, β-lactamase, caroxblesterase HCE1, peroxidase enxyme. In some embodiments, an anti-cancer agent is an pro-apoptotic gene, or a fragment or variant thereof.

In some embodiments, an pro-apoptotic agents expressed by the ASC is a nucleic acid agent, for example a nucleic acid molecule such as a RNAi molecule or antisense oligonucleotide. In some embodiments, a RNA interference (RNAi) agent useful in the methods as disclosed herein includes, for example but is not limited to antisense nucleotide acid, oligonucleotide, siRNA, shRNA, miRNA, ribozyme, avimirs or variants or derivatives thereof. In some embodiments, a RNAi agent is a shRNA molecule. In such an embodiment, a RNAi molecule would gene silence an essential gene, as that term is defined herein (e.g. a gene encoding a protein necessary for cell survival) or alternatively a cancer target gene. Examples of cancer target genes gene silenced by a RNAi anti-cancer agent include, for example, but are no limited to HER2/Her-2, BRAC1 and BRAC2, Rb, p53 and the like. Designing siRNA molecules against any of these proteins is routine to a skilled artisan.

In some embodiments, the anti-cancer agent is a RNAi agent which gene silences a cancer-causing oncogene in a subject, where the subject comprises both a oncogene (i.e. an abnormal cancer-causing gene) and a normal gene. The cancer-causing gene is different from the normal copy of the gene by a difference in the polynucleotide sequence. Such cancer-causing genes are well known in the art and are encompassed for being gene silenced by an anti-cancer agent as disclosed herein, and include, for example, but are not limited to, oncogenes such as ABL1, BCL1, BCL2, BCL6, CBFA2, CBL, CSF1R, ERBA, ERBB, EBRB2, FGR, FOS, FYN, HRAS, JUN, LCK, LYN, MYB, MYC, NRAS, RET or SRC; tumor suppressor genes such as BRCA1 or BRCA2; adhesion molecules; cyclin kinases and their inhibitors. An exemplary list of potential target genes, including developmental genes, oncogenes, and enzymes, and a list of cancers that can be treated according to the present invention can be found in WO 99/32619 which is incorporated herein in its entirety by reference.

In some embodiments, an anti-cancer agent is a RNAi anti-cancer agent which selectively targets, and preferably eliminates a cancer stem cell. Such RNAi anti-cancer agents are known in the art, and include RNAi anti-cancer-agents which gene silence a gene expressed specifically in a cancer stem cell.

In some embodiments, the nucleic acid sequence encoding an anti-cancer agent enables expression of the anti-cancer agent specially to kill cancer cells, for example it may encode an immunotoxin molecule and the like.

In some embodiments, an anti-cancer agent specifically excludes any anti-angiogenic agents or any agents which inhibits the formation of a blood vessels or inhibits angiogenesis for example migration of endothelial cells.

In another embodiments, the anti-cancer agent specifically excludes hormones or hormone steroids. In some embodiments, the excluded hormones include a cytokine, or growth hormone such as human growth factor, fibroblast growth factor, nerve growth factor, insulin-like growth factors, hemopoietic stem cell growth factors, members of the fibroblast growth factor family, members of the platelet-derived growth factor family, vascular and endothelial cell growth factors, members of the TGFb family (including bone morphogenic factor), acidic and basic fibroblast growth factors, vascular endothelial growth factor (VEGF), epidermal growth factor (EGF), transforming growth factor a (TGFa) and .beta (TGFb), platelet-derived endothelial growth factor (PDEGF), platelet-derived growth factor (PDGF), tumor necrosis factor .alpha (TNFα), hepatocyte growth factor (HGF), insulin like growth factor, erythropoietin (EPO), colony stimulating factor (CSF), macrophage-CSF, granulocyte/macrophage CSF and nitric oxide synthase (NOS) or enzymes specific for congenital disorders (e.g., dystrophic).

In another embodiments, the anti-cancer agent specifically excludes angiogenic hormones (e.g., vascular growth factor, vascular and endothelial cell growth factor, etc.), which induce angiogenesis within tissues or promotes neovascularization within the tissue.

In some embodiments, an anti-cancer agent is a pro-apoptotic agent. In some embodiments, the pro-apoptotic agent is a RNAi agent. In some embodiments, an RNAi pro-apoptotic agent gene silences an anti-apoptotic molecule (also known as survival molecules or survival factors). Examples of such molecules include, but are not limited to numerous anti-apoptotic molecules which are well known by person of ordinary skill in the art, for example Bcl-2; Bcl-XL; Hsp27; inhibitors of apoptosis (IAP) proteins.

In some embodiments, an pro-apoptotic agent expressed by the ASC is a protein (e.g. an enzyme) which activates a second agent (i.e. a pro-drug). For example, but not wishing to be bound by theory, an anti-cancer agent can be a tyrosine kinase, for example β glucuronidase activity. β-Glucuronidase activates the low-toxic prodrugs such as 9-aminocamptothecin and p-hydroxy aniline mustard, or analogue such as a N-[4-doxorubicin-N-carbonyl (oxymethyl phenyl]O-β-glucuronyl carbamate (DOX-GA3) have been developed to improve the antitumor effects of doxorubicin (DOX). The prodrug DOX-GA3 was initially designed to be activated into an active molecule or drug by human β-glucuronidase (GUS) to result in a highly cytotoxic effect specifically in the tumor site. The potency of such prodrugs can also be greatly enhanced with the incorporation of an appropriate radionuclide in a combined chemo- and radio-therapy of anti-cancer (CCRTC) strategy. In some embodiments, the prodrug can also be utilized to modify liposomes for efficient delivery of anti-cancer drugs (Chen et al, current medicinal chemistry; 2003 3; 139-150; Chen et al, cancer Gene Ther, 2006;).

In another embodiment, an pro-apoptotic agent expressed by the ASC is for example, hypoxanthine-guanine phosphoribosyltransferase (HGPRT), from the parasite Trypanosoma brucei (Tb), which can convert allopurinol, a purine analogue, to corresponding nucleotides with greater efficiency than its human homologue, therefore is capable of activating the prodrug allopurinol to a cytotoxic metabolite (Trudeau et al, 2001; Human Gene Ther; 12:1673-1680). In another embodiment, the effector molecule can be the bacterial nitrobenzene nitroreductase (NbzA) from Pseudomonas pseudoalcaligenes JS45, which activates the dinitrobenzamide cancer prodrug CB1954 and the proantibiotic nitrofurazone (Berne et al, 2006; Biomacromolecules, 7; 2631-6).

In another embodiment, an pro-apoptotic agent expressed by the ASC is β-lactamase, which produces active agents or drugs from the pro-drug desacetylvinblastine-3-carboxylic acid hydrazide (DAVLBHYD) or other analogues. In such and embodiment, the Enterobacter cloacae beta-lactamase (bL) as an effector protein can activate the anticancer prodrugs 7-(4-carboxybutanamido)cephalosporin mustard (CCM), a cephalosporin prodrug of phenylenediamine mustard (PDM) (Svensson et al, 1999; J Med. Chem., 41:1507-12).

In another embodiment, an pro-apoptotic agent expressed by the ASC can be a molecule that catalyzes an antiviral drug, for example, but not limited to Oseltamivir which is commonly used as an anti-viral drug can act as a secondary agent for carboxylesterase HCE1 as an effector molecule (Shi et al, 2006; J Pharmacol Exp Ther.).

In one embodiment, a prodrug/enzyme combinations known in the art can be used as the pro-apoptotic agent expressed by the ASC and are encompassed for use in the methods as disclosed herein, including enzymes that produce toxic radicals on photodynamic therapy (see Wardman et al, 2001; Scientific Yearbook, 2001-2002), for example peroxidase genes can be used as effector molecules.

Expression Systems

In some embodiments, an anti-cancer agent is expressed from a ASC, where the anti-cancer agent is encoded by a nucleic acid sequence operatively linked to a promoter. Thus, the activation of the promoter results in expression of the anti-cancer agent. Specific promoters can be used as disclosed herein, for example, constitutive promoters as well as inducible promoters to selectively turn on or off the expression of the anti-cancer agent.

Promoters are well known by persons of ordinary skill in the art, and any suitable promoter can be used for the expression of the anti-cancer agent. In some embodiments, a promoter is a constitutive promoter or fragments or derivatives thereof. In alternative embodiments, a promoter is a tissue-specific or cell-specific promoter, or fragments or derivatives thereof.

In some embodiments, the nucleic acid sequence encoding the anti-cancer agent as disclosed herein can optionally further comprise a promoter, for example but not limited to, enhancers, 5′ untranslated regions (5′UTR), 3′ untranslated regions (3′UTR), and repressor sequences; constitutive promoters, inducible promoter; tissue specific promoter, cell-specific promoter or variants thereof. Examples of tissue-specific promoters which can be used include, but are not limited to, albumin, lymphoid specific promoters, T-cell promoters, neurofilament promoter, pancreas specific promoters, milk whey promoter; hox promoters, α-fetoprotein promoter, human LIMK2 gene promoters, FAB promoter, insulin gene promoter, transphyretin, alpha.1-antitrypsin, plasminogen activator inhibitor type 1 (PAI-1), apolipoprotein myelin basic protein (MBP) gene, GFAP promoter, OPSIN promoter, NSE, Her2, erb2, and fragments and derivatives thereof. Examples of other promoters which can be used include, but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and variants thereof.

In some embodiments, the ASC can optionally be transduced with a second nucleic acid sequence comprising a nucleotide sequence encoding at least one marker gene which is operatively linked to a second promoter wherein gene expression of the marker gene identifies cells also expressing the target gene. The first and second nucleic acids can be in the same or different vectors.

In some embodiments, the nucleotide construct optionally further comprises a nucleotide sequence encoding at least one marker gene, wherein the expression of the marker gene is operatively linked to the first repressor promoter sequence, wherein gene expression of the marker gene identifies cells also expressing the target gene. In some embodiments, the nucleic acid sequence can optionally further comprises at least one nucleotide sequence selected from the group of; internal ribosome target site (IRES) and/or multiple cloning nucleotide sequence site. Alternatively, in some embodiments, the nucleic acids and systems as disclosed herein further comprises a marker gene or a fragment thereof operatively linked to the first repressor promoter sequence.

Promoters

The term “promoter” as used herein refers to any sequence that regulates the expression of a nucleic acid sequence, such as, the heterologous target gene, RNAi agent or nucleic acids encoding repressor proteins. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. The phrases “operable linked,” “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

A. Repressible and Inducible Promoters

(i) Inducible Promoters

Inducible promoters are characterized by resulting in additional transcription activity when in the presence of, influenced by, or contacted by the inducer than when not in the presence of, under the influence of, or in contact with the promoter. The inducer may be endogenous, or a normally exogenous compound or protein that is administered in such a way as to be active in inducing expression from the inducible promoter. In some embodiments, the inducer agent, i.e. a compound or protein, can itself be the result of transcription or expression of a polynucleotide (i.e. can be a repressor protein), which itself may be under the control or an inducible or repressible promoter. Examples of inducible promoters include but are not limited to; tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

Inducible promoters useful in the methods and systems of the present invention are capable of functioning in a eukaryotic host organism. Preferred embodiments include mammalian inducible promoters, although inducible promoters from other organisms as well as synthetic promoters designed to function in a eukaryotic host may be used. The important functional characteristic of the inducible promoters of the present invention is their ultimate inducibility by exposure to an externally applied agent, such as an environmental inducing agent. Appropriate environmental inducing agents include exposure to heat, various steroidal compounds, divalent cations (including Cu⁺² and Zn⁺²), galactose, tetracycline, IPTG (isopropyl-β-D thiogalactoside), as well as other naturally occurring and synthetic inducing agents and gratuitous inducers.

The nucleic acid construct and systems disclosed herein encompass the inducibility of a eukaryotic promoter by either of two mechanisms. In particular embodiments of the present invention, the nucleic acid construct comprises suitable inducible promoters can be dependent upon transcriptional activators that, in turn, are reliant upon an environmental inducing agent. In some embodiments, the inducible promoters can be repressed by a transcriptional repressor which itself is rendered inactive by an environmental inducing agent. Thus the inducible promoter can be either one that is induced by an environmental agent that positively activates a transcriptional activator, or one which is derepressed by an environmental agent which negatively regulates a transcriptional repressor. For example, as demonstrated in the Examples, one inducible promoter used is the LacO system, whereby addition of the externally added agent IPTG negatively regulates the transcriptional repressor LacI.

Inducible promoters useful in the methods and systems as disclosed herein include those controlled by the action of latent transcriptional activators that are subject to induction by the action of environmental inducing agents. Preferred examples include the copper-inducible promoters of the yeast genes CUP1, CRS5, and SOD1 that are subject to copper-dependent activation by the yeast ACE1 transcriptional activator (see e.g. Strain and Culotta, 1996; Hottiger et al., 1994; Lapinskas et al., 1993; and Gralla et al., 1991). Alternatively, the copper inducible promoter of the yeast gene CTT1 (encoding cytosolic catalase T), which operates independently of the ACE1 transcriptional activator (Lapinskas et al., 1993), can be utilized. The copper concentrations required for effective induction of these genes are suitably low so as to be tolerated by most cell systems, including yeast and Drosophila cells. Alternatively, other naturally occurring inducible promoters can be used in the present invention including: steroid inducible gene promoters (see e.g. Oligino et al. (1998) Gene Ther. 5: 491-6); galactose inducible promoters from yeast (see e.g. Johnston (1987) Microbiol Rev 51: 458-76; Ruzzi et al. (1987) Mol Cell Biol 7: 991-7); and various heat shock gene promoters. Many eukaryotic transcriptional activators have been shown to function in a broad range of eukaryotic host cells, and so, for example, many of the inducible promoters identified in yeast can be adapted for use in a mammalian host cell as well. For example, a unique synthetic transcriptional induction system for mammalian cells has been developed based upon a GAL4-estrogen receptor fusion protein that induces mammalian promoters containing GAL4 binding sites (Braselmann et al. (1993) Proc Natl Acad Sci USA 90: 1657-61). These and other inducible promoters responsive to transcriptional activators that are dependent upon specific inducing agents are suitable for use with the present invention.

Inducible promoters useful in the methods and systems as disclosed herein also include those that are repressed by repressors that are subject to inactivation by the action of environmental inducing agents. Examples include prokaryotic repressors that can transcriptionally repress eukaryotic promoters that have been engineered to incorporate appropriate repressor-binding operator sequences. Preferred repressors for use in the present invention are sensitive to inactivation by physiologically benign inducing agent. Thus, where the lac repressor protein is used to control the expression of a eukaryotic promoter that has been engineered to contain a lacO operator sequence, treatment of the host cell with IPTG will cause the dissociation of the lac repressor from the engineered promoter and allow transcription to occur. Similarly, where the tet repressor is used to control the expression of a eukaryotic promoter that has been engineered to contain a tetO operator sequence, treatment of the host cell with tetracycline will cause the dissociation of the tet repressor from the engineered promoter and allow transcription to occur.

An inducible promoter useful in the methods and systems as disclosed herein can be induced by one or more physiological conditions, such as changes in pH, temperature, radiation, osmotic pressure, saline gradients, cell surface binding and the concentration of one or more extrinsic or intrinsic agents. The extrinsic agent may comprise amino acids and amino acid analogs, saccharides and polysaccharides, nucleic acids, transcriptional activators and repressors, cytokines, toxins, petroleum-based compounds, metal containing compounds, salts, ions, enzyme substrate analogs and combinations thereof. In specific embodiments, the inducible promoter is activated or repressed in response to a change of an environmental condition, such as the change in concentration of a chemical, metal, radiation or nutrient or change in pH.

Furthermore, an inducible promoter useful in the methods and systems as disclosed herein can be a phage inducible promoter, nutrient inducible promoter, temperature inducible promoter, radiation inducible promoter, metal inducible promoter, hormone inducible promoter, steroid inducible promoter, and/or hybrids and combinations thereof. Promoters that are inducible by ionizing radiation can be used in certain embodiments, particularly in gene therapy of cancer, where gene expression is induced locally in the cancer cells by exposure to ionizing radiation such as UV or x-rays. Radiation inducible promoters include the non-limiting examples of fos promoter, c-jun promoter or at least one CArG domain of an Egr-1 promoter. Examples of inducible promoters include promoters from genes such as cytochrome P450 genes, heat shock protein genes, metallothionein genes, hormone-inducible genes, such as the estrogen gene promoter, and such.

In further embodiments, an inducible promoter useful in the methods and systems as disclosed herein can be Zn²⁺ metallothionein promoter, metallothionein-1 promoter, human metallothionein IIA promoter, lac promoter, lacO promoter, mouse mammary tumor virus early promoter, mouse mammary tumor virus LTR promoter, triose dehydrogenase promoter, herpes simplex virus thymidine kinase promoter, simian virus 40 early promoter or retroviral myeloproliferative sarcoma virus promoter.

Examples of inducible promoters include mammalian probasin promoter, lactalbumin promoter, GRP78 promoter, or the bacterial tetracycline-inducible promoter. Other examples include heat shock, steroid hormone, heavy metal, phorbol ester, adenovirus E1A element, interferon, and serum inducible promoters.

Inducible promoters useful in the methods and systems as disclosed herein for in vivo uses may include those responsive to biologically compatible agents, such as those that are usually encountered in defined animal tissues. An example is the human PAI-1 promoter, which is inducible by tumor necrosis factor. Further suitable examples cytochrome P450 gene promoters, inducible by various toxins and other agents; heat shock protein genes, inducible by various stresses; hormone-inducible genes, such as the estrogen gene promoter, and such.

Inducible promoters may be inducible by Cu²⁺, Zn²⁺, tetracycline, tetracycline analog, ecdysone, glucocorticoid, tamoxifen, or an inducer of the lac operon (LacO). The promoter may be inducible by ecdysone, glucocorticoid, or tamoxifen. In specific embodiments, the inducible promoter is a phage inducible promoter, nutrient inducible promoter, temperature inducible promoter, radiation inducible promoter, metal inducible promoter, hormone inducible promoter, steroid inducible promoter, or combination thereof. Examples of radiation inducible promoters include fos promoter, jun promoter, or erg promoter.

Systems for the regulation of gene expression that may be used within the contemplated scope of the invention include regulatory systems utilizing compounds such as progesterone, estrogen, and/or ecdysone.

An expression constructs may also contain a chemically inducible promoter (review article: Gatz et al. (1997) Annu Rev Plant Physiol Plant MoI Biol 48:89-108), by means of which the expression of the heterologous target gene can be controlled at a particular point in time. Such promoters such as, for example, a salicylic acid-inducible promoter (WO 95/19443), a benzenesulfonamide-inducible promoter (EP 0 388 186), a tetracycline-inducible promoter (Gatz et al. (1991) MoI Gen Genetics 227:229-237), an abscisic acid-inducible promoter EP 0 335 528) or an ethanol-cyclohexanone-inducible promoter (WO 93/21334) can likewise be used. Also suitable is the promoter of the glutathione-S transferase isoform II gene (GST-II-27), which can be activated by exogenously applied safeners such as, for example, N,N-diallyl-2,2-dichloroacetamide (WO 93/01294) and which is operable in a large number of tissues of both monocotyledonous and dicotyledonous. Further exemplary inducible promoters that can be utilized in the instant invention include that from the ACE1 system which responds to copper (Mett et al. PNAS 90: 4567-4571 (1993)). An exemplary inducible promoter is the inducible promoter from a steroid hormone gene, the transcriptional activity of which is induced by a glucocorticosteroid hormone (Schena et al. (1991) Proc Nat'l Acad Sci USA 88:10421).

(ii) Repressor Promoters

Promoters contemplated in the invention include conditional promoters, often referred to herein as “repressor promoters”. A conditional promoter or a repressor promoter is a promoter that is active only under certain conditions. For example, the promoter may be inactive or repressed when a particular repressor agent, such as a chemical compound, or particular protein referred to as a “repressor protein” is present. When the agent is no longer present, transcription is activated or de-repressed. Examples of conditional promoters may include the promoter Met25 (Kerjan P. et al., 1986), which can be regulated as a function of methionine concentration, or the promoters GAL1 or GAL10 (Johnston and Davis, 1984), which can be regulated as a function of galactose concentration, but are not limited to such.

A repressible promoter is one whose ability to promote transcription is at least partially responsive to the presence or action of a repressor, which is a compound or protein that acts to repress the promoter and so reduce, inhibit, or repress transcription of the polynucleotide under the influence of the promoter. Repressible promoters are characterized by resulting in lower levels of transcription activity when in the presence of, influenced by, or contacted by the repressor than when not in the presence of, under the influence of, or in contact with the promoter. The repressor may be endogenous, or a normally exogenous compound or protein that is administered in such a way as to be active in repressing the repressible promoter. Provision of the repressor, i.e. a compound or protein, may itself be the result of transcription or expression of a polynucleotide, which itself may be under the control or an inducible or repressible promoter.

In specific embodiments, the nucleic acid construct as disclosed herein comprises a repressor promoter which transcriptionally controls the expression of the RNAi agent of the nucleic acid construct. In some embodiments, the expression of the repressor protein to the repressor promoter can be transcriptionally controlled by an inducible promoter or a constitutive promoter.

In some embodiments, promoters that are controllable by an external stimulus are utilized in methods and compositions of the present invention. Examples of promoters that are controllable by external stimulus include, for example, the P_{L} promoter, P_{R} promoter, Pᵣₑ promoter, Pᵣₘ promoter, P′_{R} promoter, T₇ late promoters, trp promoter, tac promoter, lac promoter, gal promoter, ara promoter or recA promoter or fragments thereof. In particular embodiments, operator sequences from these promoters are utilized in the nucleic acid construct and systems of the present invention.

In particular embodiments of the present invention, control of expression is generated through the use of a particular system comprising both polynucleotide and polypeptide components. In both prokaryotes and eukaryotes, polypeptides having affinity for specific sites on DNA modulate transcriptional expression of genes, and interaction with DNA at specific sites in genes, the polypeptides, herein referred to “repressor proteins” or “repressors,” hinder transcription by, for example, making the DNA inaccessible to RNA polymerase.

Typically, the LTRi construct as disclosed herein comprises an RNAi agent operatively linked to at least one tet operator (tetO) sequence. The tetO sequence can be obtained, for example, according to Hillen & Wissmann, “Topics in Molecular and Structural Biology,” in Protein-Nucleic Acid Interaction, Saeger & Heinemann, eds., Macmillan, London, 1989, Vol. 10, pp. 143-162, the contents of which are fully incorporated by reference herein. Other tetO sequences that may be used in the practice of the invention may be obtained from Genbank and/or are disclosed in Waters, S. H. et al. (1983) Nucl. Acids Res. 11:6089-6105; Hillen, W. and Schollmeier, K. (1983) Nucl. Acids Res. 11:525-539; Stuber, D. and Bujard, H. (1981) Proc. Natl. Acad. Sci. USA 78:167-171; Unger, B. et al. (1984) Nucl Acids Res. 12:7693-7703; and Tovar, K. et al. (1988) Mol. Gen. Genet. 215:76-80, which are fully incorporated by reference herein in their entirety. One, two, three, four, five, six, seven, eight, nine or ten or more copies of the tet operator sequence may be employed, with a greater number of such sequences allowing an enhanced range of regulation, in some embodiments.

However, other repressor domains include ERD or SID transcriptional repressor domains can be used, for example, transcription factors and transcription factor domains that act as transcriptional repressors include, for example, MAD (see, e.g., Sommer et al., 1998; Gupta et al., 1998; Queva et al., 1998; Larsson et al., 1997; Laherty et al., 1997; and Cultraro et al., 1997); FKHR (forkhead in rhapdosarcoma gene; Ginsberg et al., 1998; Epstein et al., 1998); EGR-1 (early growth response gene product-1; Yan et al., 1998; and Liu et al., 1998); the ets2 repressor factor repressor domain (ERD; Sgouras et al, 1995); and the MAD smSIN3 interaction domain (SID; Ayer et al., 1996).

DNA-binding proteins have been characterized extensively to determine how these polypeptides actually contact the DNA molecule, for those embodiments concerning repression through direct binding mechanisms, and interact with it to influence gene expression. Some non-limiting examples of these polypeptides include those that comprise the structural motif alpha-helix-turn-alpha-helix (H-T-H). These proteins bind as dimers or tetramers to DNA at specific operator sequences that have approximately palindromic sequences. Contacts made by two adjacent alpha helices of each monomer in and around two sites in the major groove of B-form DNA are a major feature in the interface between DNA and these proteins. Proteins that bind in this manner share sequence similarity in the H-T-H region but vary in the extent of similarity in other regions. This group of proteins includes, for example, the temperate bacteriophage repressor proteins and Cro proteins, bacterial metabolic repressor proteins such as GalR, LacI, LexA, and TrpR, bacterial activator protein CAP and dual activator/repressor protein AraC, bacterial transposon and plasmid TetR proteins, the yeast mating type regulator proteins MATa1 and MATalpha2 and eukaryotic homeobox proteins.

Other repressors have little or no sequence homology to H-T-H binding proteins and have no H-T-H binding motif. Binding of operators with approximate palindromic sequence symmetry is observed among some proteins of this group, such as Salmonella typhimurium bacteriophage P22 Mnt protein (VERS87a) and E. coli TyrR repressor protein (DEFE86). Others of this group bind to operator sequences that are partially symmetric (S. typhimurium phage P22 Arc protein, VERS87b; E. coli Fur protein, DEL087; plasmid R6K pi protein, FILU85) or non-symmetric (phage Mu repressor, KRAU86).

A skilled artisan recognizes that a repressor and/or DNA binding domain utilized in the present invention can comprise a mutation, as compared to wild-type, so long as the mutation does not deleteriously affect the respective functions of these components, and these mutated components may be utilized in methods and compositions of the present invention.

B. Inducible Agents and Inducible Systems

The administration or removal of an agent as disclosed herein results in a switch between the on or off states of the transcription of the heterologous target gene. Several small molecule ligands have been shown to mediate regulated gene expressions, either in tissue culture cells and/or in transgenic animal models. These include the FK1012 and rapamycin immunosupressive drugs (Spencer et al., 1993; Magari et al., 1997), the progesterone antagonist mifepristone (RU486) (Wang, 1994; Wang et al., 1997), the tetracycline antibiotic derivatives (Gossen and Bujard, 1992; Gossen et al., 1995; Kistner et al., 1996), and the insect steroid hormone ecdysone (No et al., 1996). All of these references are herein incorporated by reference.

By way of further example, Yao discloses in U.S. Pat. No. 6,444,871 which is incorporated herein by reference, prokaryotic elements associated with the tetracycline resistance (tet) operon, a system in which the tet repressor protein is fused with polypeptides known to modulate transcription in mammalian cells. The fusion protein has then been directed to specific sites by the positioning of the tet operator sequence. For example, the tet repressor has been fused to a transactivator (VP16) and targeted to a tet operator sequence positioned upstream from the promoter of a selected gene (Gussen et al., 1992; Kim et al., 1995; Hennighausen et al., 1995). The tet repressor portion of the fusion protein binds to the operator thereby targeting the VP16 activator to the specific site where the induction of transcription is desired. An alternative approach has been to fuse the tet repressor to the KRAB repressor domain and target this protein to an operator placed several hundred base pairs upstream of a gene. Using this system, it has been found that the chimeric protein, but not the tet repressor alone, is capable of producing a 10 to 15-fold suppression of CMV-regulated gene expression (Deuschle et al., 1995).

One example of a repressor promoter useful in the methods and systems as disclosed herein is the Lac repressor (lacR)/operator/inducer system of E. coli which and has been used to regulate gene expression by three different approaches: (1) prevention of transcription initiation by properly placed lac operators at promoter sites (Hu and Davidson, 1987; Brown et al., 1987; Figge et al., 1988; Fuerst et al., 1989; Deuschle et al., 1989; (2) blockage of transcribing RNA polymerase II during elongation by a LacR/operator complex (Deuschle et al. (1990); and (3) activation of a promoter responsive to a fusion between LacR and the activation domain of herpes simples virus (HSV) virion protein 16 (VP16) (Labow et al., 1990; Baim et al., 1991).

In one version of the Lac system, expression of lac operator-linked sequences is constitutively activated by a LacR-VP16 fusion protein and is turned off in the presence of isopropyl-β-D-thiogalactopyranoside (IPTG) (Labow et al. (1990), cited supra). In another version of the system, a lacR-VP16 variant is used which binds to lac operators in the presence of IPTG, which can be enhanced by increasing the temperature of the cells (Baim et al. (1991), cited supra). Thus, in some embodiments of the present invention, components of the Lac system are utilized. For example, a lac operator (LacO) may be operably linked to tissue specific promoter, and control the transcription and expression of the heterologous target gene and the first repressor proteins such as the Tet^{R}. Accordingly, the expression of the siRNA (and therefore gene silencing of the heterologous target gene) is inversely regulated as compared to the expression of the heterologous target expression, such that presence of IPTG results in inhibition of RNAi expression and induction of heterologous target gene expression.

Components of the tetracycline (Tc) resistance system of E. coli have also been found to function in eukaryotic cells and have been used to regulate gene expression. For example, the Tet repressor (TetR), which binds to tet operator (tetO) sequences in the absence of tetracycline and represses gene transcription, has been expressed in plant cells at sufficiently high concentrations to repress transcription from a promoter containing tet operator sequences (Gatz, C. et al. (1992) Plant J. 2:397-404). In some embodiments of the present invention, the Tet repressor system is similarly utilized.

A temperature-inducible gene regulatory system may also be used in the present invention, such as the exemplary TIGR system comprising a cold-inducible transactivator in the form of a fusion protein having a heat shock responsive regulator, rheA, fused to the VP16 transactivator (Weber et al., 2003a). The promoter responsive to this fusion thermosensor comprises a rheO element operably linked to a minimal promoter, such as the minimal version of the human cytomegalovirus immediate early promoter. At the permissive temperature of 37° C., the cold-inducible transactivator transactivate the exemplary rheO-CMVₘᵢₙ promoter, permitting expression of the target gene. At 41° C., the cold-inducible transactivator no longer transactivates the rheO promoter.

Other embodiments useful in the present invention include the erythromycin-resistance regulon from E. coli, having repressible (E_{off}) and inducible (Eₒₙ) systems responsive to macrolide antibiotics, such as erythromycin, clarithromycin, and roxithromycin (Weber et al., 2002). The E_{off} system utilizes an erythromycin-dependent transactivator, wherein providing a macrolide antibiotic represses transgene expression. In the Eₒₙ, system, the binding of the repressor to the operator results in repression of transgene expression. Therein, in the presence of macrolides gene expression is induced.

Fussenegger et al. (2000) describe repressible and inducible systems using a Pip (pristinamycin-induced protein) repressor encoded by the streptogramin resistance operon of Streptomyces coelicolor, wherein the systems are responsive to streptogramin-type antibiotics (such as, for example, pristinamycin, virginiamycin, and Synercid). The Pip DNA-binding domain is fused to a VP16 transactivation domain or to the KRAB silencing domain, for example. The presence or absence of, for example, pristinamycin, regulates the PipON and PipOFF systems in their respective manners, as described therein.

Another example of a transgene expression system utilizes a quorum-sensing (referring to particular prokaryotic molecule communication systems having diffusable signal molecules that prevent binding of a repressor to an operator site, resulting in derepression of a target regulon) system. For example, Weber et al. (2003b) employ a fusion protein comprisign the Streptomyces coelicolor quorum-sending receptor to a transactivating domain that regulates a chimeric promoter having a respective operator that the fusion protein binds. The expression is fine-tuned with non-toxic butyrolactones, such as SCB1 and MP133.

In particular embodiments, multiregulated multigene therapeutic gene expression systems that are functionally compatible with one another are utilized in the present invention (see, for example, Kramer et al. (2003)). For example, in Weber et al. (2002), the macrolide-responsive erythromycin resistance regulon system is used in conjunction with a streptogramin (PIP)-regulated and tetracycline-regulated expression systems.

In some embodiments, the inducible promoter can be a heat-inducible promoter. Any heat-inducible promoter can be used in accordance with the methods of the present invention, including but not limited to a heat-responsive element in a heat shock gene (e.g., hsp20-30, hsp27, hsp40, hsp60, hsp70, and hsp90). See Easton et al. (2000) Cell Stress Chaperones 5(4):276-290; Csermely et al. (1998) Pharmacol Ther 79(2): 129-1 68; Ohtsuka & Hata (2000) lnt J Hyperthermia 16(3):231-245; and references cited therein. Sequence similarity to heat shock proteins and heat-responsive promoter elements have also been recognized in genes initially characterized with respect to other functions, and the DNA sequences that confer heat inducibility are suitable for use in the disclosed gene therapy vectors. For example, expression of glucose-responsive genes (e.g., grp94, grp78, mortalin/grp75) (Merrick et al. (1997) Cancer Lett 119(2): 185-1 90; Kiang et al. (1998) FASEB J 12(14):1571-16-579), calreticulin (Szewczenko-Pawlikowski et al. (1997) MoI Cell Biochem 177(1-2): 145-1 52); clusterin (Viard et al. (1999) J Invest Dermatol 112(3):290-296; Michel et al. (1997) Biochem J 328(Pt1):45-50; Clark & Griswold (1997) J Androl 18(3):257-263), histocompatibility class I gene (HLA-G) (Ibrahim et al. (2000) Cell Stress Chaperones 5(3):207-218), and the Kunitz protease isoform of amyloid precursor protein (Shepherd et al. (2000) Neuroscience 99(2):31 7-325) are upregulated in response to heat.

In the case of clusterin, a 14 base pair element that is sufficient for heat-inducibility has been delineated (Michel et al. (1997) Biochem J 328(Pt1):45-50). Similarly, a two sequence unit comprising a 10- and a 14-base pair element in the calreticulin promoter region has been shown to confer heat-inducibility (Szewczenko-Pawlikowski et al. (1997) MoI Cell Biochem 177(1-2): 145-1 52). Other promoter responsive to non-heat stimuli that can be used. For example, the mortalin promoter is induced by low doses of ionizing radiation (Sadekova (1997) lnt J Radiat Biol 72(6):653-660), the hsp27 promoter is activated by 17. beta.-estradiol and estrogen receptor agonists (Porter et al. (2001) J MoI Endocrinol 26(1):31-42), the HLA-G promoter is induced by arsenite, hsp promoters can be activated by photodynamic therapy (Luna et al. (2000) Cancer Res 60(6): 1637-1 644). A suitable promoter can incorporate factors such as tissue-specific activation. For example, hsp70 is transcriptionally impaired in stressed neuroblastoma cells (Drujan & De Maio (1999) 12(6):443-448). The mortalin promoter, which is up-regulated in human brain tumors (Takano et al. (1997) Exp Cell Res 237(1):38-45). A promoter employed in methods of the present invention can show selective up-regulation in tumor cells as described, for example, for mortalin (Takano et al. (1997) Exp Cell Res 237(1):38-45), hsp27 and calreticulin (Szewczenko-Pawlikowski et al. (1997) MoI Cell Biochem 177(1-2): 145-1 52; Yu et al. (2000) Electrophoresis 2 1(14):3058-3068), grp94 and grp78 (Gazit et al. (1999) Breast Cancer Res Treat 54(2): 135-146), hsp27, hsp70, hsp73, and hsp90 (Cardillo et al. (2000) Anticancer Res 20(6B):4579-4583; Strik et al. (2000) Anticancer Res 20(6B):4457-4552).

C. Tissue-Specific Promoters

In certain aspects to the invention, a tissue-specific promoter is used to express the anti-cancer agent in a particular cell or tissue (e.g. in adipose cell). Use of tissue-specific promoters therefore provides an additional level of control of expression of the anti-cancer agent, such that, for example the anti-cancer agent is expressed only in the ASC.

Those of skill in the art of molecular biology generally know the use of promoters and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In certain embodiments, the promoters employed in the present invention are tissue-specific promoters.

Various promoters are currently used in the art to express sequences in animal, mammalian or human organism. Most of them are lacking tissue-specificity and can be advantageously combined with the teaching provided herein. For example the promoter may be selected from group consisting of the perbB2 promoter, whey acidic protein promoter, stromelysin 3 promoter, prostate specific antigen promoter, probasin promoter.

In some embodiments, other tissue specific promoters include, for example but are not limited to such as albumin (liver specific, Pinkert et al., (1987)), lymphoid specific promoters (Calame and Eaton, 1988), in particular promoters of T-cell receptors (Winoto and Baltimore, (1989)) and immunoglobulins; Banerji et al., (1983); Queen and Baltimore, 1983), neuron specific promoters (e.g. the neurofilament promoter; Byrne and Ruddle, 1989), pancreas specific promoters (Edlund et al., (1985)) or mammary gland specific promoters (milk whey promoter, U.S. Pat. No. 4,873,316 and European Application Publication No. 264,166) as well as developmentally regulated promoters such as the murine hox promoters (Kessel and Cruss, Science 249:374-379 (1990)) or the α-fetoprotein promoter (Campes and Tilghman, Genes Dev. 3:537-546 (1989)), the contents of each of which are fully incorporated by reference herein. Preferably, the promoter is constitutive in the respective cell types.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996), and the SM22.alpha. promoter. Tissue-specific promoters and/or regulatory elements will be useful in certain embodiments. Other examples of such tissue-specific promoters that may be used with the expression vectors of the invention include promoters from the liver fatty acid binding (FAB) protein gene, specific for colon epithelial cells; the insulin gene, specific for pancreatic cells; the transphyretin, .alpha.1-antitrypsin, plasminogen activator inhibitor type 1 (PAI-1), apolipoprotein AI and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells.

Also contemplated as useful in the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest. Alternatively, a tissue-specific promoter for gene therapy such as cancer gene therapy, may be employed in the present invention.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta-galactosidase alpha-2,6 sialylkansferase promoter, insulin-like growth factor binding protein (IGFBP-1) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific expression elements for gastric tissue include but are not limited to the human H+/K+-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamma enolase (neuron-specific enolase, NSE) promoter. Exemplary tissue-specific expression elements for the brain include, but are not limited to, the neurofilament heavy chain (NF-H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter.

In some embodiments, tissue-specific expression elements for breast cells are for example, but are not limited to, the human alpha-lactalbumin promoter. Exemplary tissue-specific expression elements for the lung include, but are not limited to, the cystic fibrosis transmembrane conductance regulator (CFTR) gene promoter.

Other elements aiding specificity of expression in a tissue of interest can include secretion leader sequences, enhancers, nuclear localization signals, endosmolytic peptides, etc. Preferably, these elements are derived from the tissue of interest to aid specificity. In general, the in vivo expression element shall include, as necessary, 5′ non-transcribing and 5′ non-translating sequences involved with the initiation of transcription. They optionally include enhancer sequences or upstream activator sequences.

D. Constitutive Promoters

Examples of such constitutive promoters useful in the nucleic acid sequence encoding the anti-cancer agent to be expressed by the ASC as disclosed herein includes, for example but are not limited to the human cytomegalovirus promoter IE as taught by Boshart et al., (1985), ubiquitously expressing promoters such as HSV-Tk (McKnight et al., (1984) and β-actin promoters (e.g. the human β-actin promoter as described by Ng et al., (1985)), as well as promoters in combination with control regions allowing integration site independent expression of the transgene (Grosveld et al., (1987)).

Constitutive mammalian promoters include, but are not limited to, polymerase promoters as well as the promoters for the following genes: hypoxanthine phosphoribosyl transferase (HPTR), adenosine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

E. Other Regulatory Elements

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

In some embodiments, the nucleic acid sequence encoding the anti-cancer agent to be expressed by the ASC as disclosed herein can further comprise multiple cloning site (MCS), which is a nucleic acid region that contains multiple restriction enzyme sites, any of which can be used in conjunction with standard recombinant technology to digest the vector. (See Carbonelli et al., 1999, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology. In some embodiments, multiple cloning sites, are useful in the nucleic acid constructs and compositions as disclosed herein for replacing one heterogonous target gene with another heterologous target gene.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

In some embodiments, the nucleic acid sequence encoding the anti-cancer agent to be expressed by the ASC as disclosed herein can further comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

F. Other Components of Nucleic Acid Sequence Encoding the Anti-Cancer Agent

A. Reporter Genes and Selectable Marker Genes

In certain embodiments of the invention, ASC containing the nucleic acid sequence encoding the anti-cancer agent to be expressed as disclosed herein can be identified in vitro or in vivo by including a marker in the nucleic acid construct. Such markers would confer an identifiable change to the ASC permitting easy identification of ASCs containing the expression vector and/or expressing the anti-cancer agent. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Where the reporter gene is to identify ASC expressing the anti-cancer agent, the location of the reporter gene and/or selectable marker can be downstream (i.e. 3′) of the nucleic acid encoding the anti-cancer agent and is under the same transcriptional control (i.e. promoter) of the expression of the anti-cancer agent, so when the expression of the anti-cancer agents occurs, expression of the reporter gene also occurs.

In further embodiments of the present invention, the nucleic acid sequence encoding the anti-cancer agent can further comprise a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Reporter genes encode readily quantifiable proteins and, via their color or enzyme activity, make possible an assessment of the transformation efficacy, the site of expression or the time of expression. In some embodiments, the following genes encoding reporter proteins are useful; (Schenborn and Groskreutz (1999) MoI Biotechnol 13(1):29-44) such as the green fluorescent protein (GFP) (Haseloff et al. (1997) Proc Natl Acad Sci USA 94(6):2122-2127; Sheen et al. (1995) Plant J 8(5):777-784; Reichel et al. 1996) Proc Natl Acad Sci USA 93(12):5888-5893; Chui et al. (1996) Curr Biol 6:325-330; Leffel et al. (1997) Biotechniques. 23(5):912-8; Tian et al. (1997) Plant Cell Rep 16:267-271; WO 97/41228), chloramphenicol transferase, a luciferase (Millar et al. (1992) Plant MoI Biol Rep 10:324-414; Ow et al. (1986) Science 234:856-859), the aequorin gene (Prasher et al. (1985) Biochem Biophys Res Commun 126(3): 1259-1268), b-galactosidase, R locus gene (encoding a protein which regulates the production of anthocyanin pigments (red coloring) in plant tissue and thus makes possible the direct analysis of the promoter activity without addition of further auxiliary substances or chromogenic substrates (Dellaporta et al. (1988) In: Chromosome Structure and Function: Impact of New Concepts, 18th Stadler Genetics Symposium, 11:263-282; Ludwig et al. (1990) Science 247:449), with b-glucuronidase (GUS) being very especially preferred (Jefferson (1987b) Plant MoI. Bio. Rep., 5:387-405; Jefferson et al. (1987) EMBO J. 6:3901-3907). b-glucuronidase (GUS) expression is detected by a blue color on incubation of the tissue with 5-bromo-4-chloro-3-indolyl-b-D-glucuronic acid, bacterial luciferase (LUX) expression is detected by light emission; firefly luciferase (LUC) expression is detected by light emission after incubation with luciferin; and galactosidase expression is detected by a bright blue color after the tissue was stained with 5-bromo-4-chloro-3-indolyl-b-D-galactopyranoside. Reporter genes may also be used as scorable markers as alternatives to antibiotic resistance markers. Such markers are used to detect the presence or to measure the level of expression of the heterologous target gene.

Introducing the Nucleic Acid Sequence Encoding the Anti-Cancer Agent into the ASC

Transfection is the introduction of nucleic acid construct as disclosed herein into a recipient cell, such as an adipose-derived stromal cell (ASC). In some embodiments the ASC is from a subject who has cancer. Efficient transfection requires vectors which facilitate the introduction of the nucleic acid construct into the desired cells, and in some embodiments may provide mechanisms for chromosomal integration, and provide for the appropriate expression of the traits or proteins encoded by those nucleic acids. The design and construction of efficient, reliable, and safe vectors for cell transfection is well known to the art. In the context of the present invention, any vector which can mediate the delivery and genomic integration of the nucleic acid construct as disclosed into the target cell, tissue or organism is contemplated to be within the scope of the invention.

Viral Vectors:

The present invention also provides methods to transduce an ASC with a nucleic acid construct or vector comprising a nucleic acid sequence encoding an anti-cancer gene operatively linked to a promoter.

The vector can be a viral vector or a non-viral vector. Suitable viral vectors include adenoviruses, adeno-associated viruses (AAVs), retroviruses, pseudotyped retroviruses, herpes viruses, vaccinia viruses, Semiliki forest virus, and baculoviruses. Suitable nonviral vectors comprise plasmids, water-oil emulsions, polethylene imines, dendrimers, micelles, microcapsules, liposomes, and cationic lipids. Polymeric carriers for gene therapy constructs can be used as described in Goldman et al (1997) Nat Biotechnol 15:462 and U.S. Pat. Nos. 4,551,482 and 5,714,166. Peptide carriers are described in U.S. Pat. No. 5,574,172.

Where appropriate, two or more types of vectors can be used together. For example, a plasmid vector can be used in conjunction with liposomes. In some embodiments of the present invention, the nucleic acid construct as disclosed herein uses of an adenovirus, a plasmid, or a liposome, each described further herein below.

As desired, vectors, especially viral vectors, can be selected to achieve integration of the nucleic acid of the construct of the invention, into the genome of the cells to be transformed or transfected. Including a ligand in the complex having affinity for a specific cellular marker can also enhance delivery of the complexes to a target in vivo.

Ligands include antibodies, cell surface markers, viral peptides, and the like, which act to home the complexes to tumor vasculature or endothelial cells associated with tumor vasculature, or to tumor cells themselves. A complex can comprise a construct or a secreted therapeutic polypeptide encoded by a construct. An antibody ligand can be an antibody or antibody fragment specific towards a tumor marker such as Her2/neu (verb-b2 avian erythroblastic leukemia viral oncogene homologue 2), CEA (carcinoembryonic antigen), ferritin receptor, or a marker associated with tumor vasculature (integrins, tissue factor, or beta.-fibronectin isoform). Antibodies or other ligands can be coupled to carriers such as liposomes and viruses, as is known in the art. See, e.g., Neri et al. (1997) Nat BioTechnology 15:1271; Kirpotin et al. (1997) Biochemistry 36:66; Cheng (1996) Human Gene Therapy 7:275; Pasqualini et al. (1997) Nat Biotechnology 15:542; Park et al. (1997) Proc Am Ass Cane Res 38:342 (1997); Nabel (1997) “Vectors for Gene Therapy” in Current Protocols in Human Genetics on CDROM, John Wiley & Sons, New York, N.Y.; U.S. Pat. No. 6,071,890; and European Patent No. 0 439 095. Alternatively, pseudotyping of a retrovirus can be used to target a virus towards a particular cell (Marin et al. (1997) MoI Med Today 3:396).

Viral vectors of the invention are preferably disabled, e.g. replication-deficient. That is, they lack one or more functional genes required for their replication, which prevents their uncontrolled replication in vivo and avoids undesirable side effects of viral infection. Preferably, all of the viral genome is removed except for the minimum genomic elements required to package the viral genome incorporating the therapeutic gene into the viral coat or capsid. For example, it is desirable to delete all the viral genome except the Long Terminal Repeats (LTRs) or Invented Terminal Repeats (ITRs) and a packaging signal. In the case of adenoviruses, deletions are typically made in the E1 region and optionally in one or more of the E2, E3 and/or E4 regions. In the case of retroviruses, genes required for replication, such as env and/or gag/pol can be deleted.

Deletion of sequences can be achieved by recombinant means, for example, involving digestion with appropriate restriction enzymes, followed by religation. Replication competent self-limiting or self-destructing viral vectors can also be used. Nucleic acid constructs of the invention can be incorporated into viral genomes by any suitable means known in the art. Typically, such incorporation will be performed by ligating the construct into an appropriate restriction site in the genome of the virus. Viral genomes can then be packaged into viral coats or capsids by any suitable procedure. In particular, any suitable packaging cell line can be used to generate viral vectors of the invention. These packaging lines complement the replication-deficient viral genomes of the invention, as they include, typically incorporated into their genomes, the genes which have been deleted from the replication-deficient genome. Thus, the use of packaging lines allows viral vectors of the invention to be generated in culture. Suitable packaging lines for retroviruses include derivatives of PA317 cells, .psi.-2 cells, CRE cells, CRIP cells, E-86-GP cells, and 293GP cells. Line 293 cells can be used for adenoviruses and adeno-associated viruses.

Suitable methods for introduction of a gene therapy construct into cells include direct injection into a cell or cell mass, particle-mediated gene transfer, electroporation, DEAE-Dextran transfection, liposome-mediated transfection, viral infection, and combinations thereof. A delivery method is selected based considerations such as the vector type, the toxicity of the encoded gene, and the condition to be treated.

Viruses of many types have formed the basis for vectors. Virus infection involves the introduction of the viral genome into the host cell. That property is co-opted for use as a gene delivery vehicle in viral based vectors. The viruses used are often derived from pathogenic viral species that already have many of the necessary traits and abilities to transfect cells. However, not all viruses will successfully transfect all cell types at all stages of the cell cycle. Thus, in the development of viral vectors, viral genomes are often modified to enhance their utility and effectiveness for introducing transgene constructs (transgenes) or other nucleic acids. At the same time, modifications may be introduced that reduce or eliminate their ability to cause disease. Thus, viral vectors derived from viruses such as retrovirus, vaccinia virus (Ridgeway, 1988; Baichwal and Sugden, 1986; Coupar et al., 1988); adeno-associated virus (AAV) (Ridgeway, 1988; Baichwal and Sugden, 1986; Hermonat and Muzycska, 1984); and herpesviruses may be employed in the present invention. They offer several attractive features for various mammalian cells (Friedmann, 1989; Ridgeway, 1988; Baichwal and Sugden, 1986; Coupar et al., 1988; Horwich et al., 1990). Other viral vectors derived from viruses such as vaccinia virus (Ridgeway, 1988; Baichwal and Sugden, 1986; Coupar et al., 1988), sindbis virus, cytomegalovirus and herpes simplex virus may also be employed.

Vectors for transduction of a nucleic acid sequence encoding an anti-cancer gene operatively linked to a promoter to a ASC are well known in the art. While overexpression using a strong non-specific promoter, such as a CMV promoter, can be used, it can be helpful to include a tissue- or cell-type-specific promoter on the expression construct—for example, the use of an adipose cell promoter or other cell-type-specific promoter can be advantageous. Viral vectors are well known to those skilled in the art and discussed in more detail herein.

These vectors are readily adapted for use in the methods of the present invention. By the appropriate manipulation using recombinant DNA/molecular biology techniques to insert an operatively linked nucleic acid sequence encoding the gene to be expressed (e.g. an anti-cancer agent, such as a pro-apoptotic gene such as TRAIL, BAX, or nucleic acid inhibitors such as a RNAi agent to an oncogene and other anti-cancer agents as disclosed herein) into the selected expression/delivery vector, many equivalent vectors for the practice of the methods described herein can be generated. It will be appreciated by those of skill in the art that cloned genes readily can be manipulated to alter the amino acid sequence of a protein.

Examples of expression vectors and host cells are the pET vectors (NOVAGEN®), pGEX vectors (GE Life Sciences), and pMAL vectors (New England labs. Inc.) for protein expression in E. coli host cell such as BL21, BL21(DE3) and AD494(DE3) pLysS, Rosetta (DE3), and Origami (DE3) ((NOVAGEN®); the strong CMV promoter-based pcDNA3.1 (INVITROGEN™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (CLONTECH®), pAd/CMV/V5-DEST, pAd-DEST vector (INVITROGEN™ Inc.) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (STRATAGENE®) for adeno-associated virus-mediated gene transfer and expression in mammalian cells; BACpak6 baculovirus (CLONTECH®) and pFastBac™ HT (INVITROGEN™ Inc.) for the expression in Spodopera frugiperda 9 (Sf9) and Sf11 insect cell lines; pMT/BiP/V5-His (INVITROGEN™ Inc.) for the expression in Drosophila Schneider S2 cells; Pichia expression vectors pPICZα, pPICZ, pFLDα and pFLD (INVITROGEN™ Inc.) for expression in Pichia pastoris and vectors pMETα and pMET for expression in P. methanolica; pYES2/GS and pYD1 (INVITROGEN™ Inc.) vectors for expression in yeast Saccharomyces cerevisiae. Recent advances in the large scale expression heterologous proteins in Chlamydomonas reinhardtii are described by Griesbeck C. et. al. 2006 Mol. Biotechnol. 34:213-33 and Fuhrmann M. 2004, Methods Mol. Med. 94:191-5. Foreign heterologous coding sequences are inserted into the genome of the nucleus, chloroplast and mitochodria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. Biolistic gene gun method is used to introduced the vector in the algae. Upon its entry into chloroplasts, the foreign DNA is released from the gene gun particles and integrates into the chloroplast genome through homologous recombination.

In one embodiment, the expression vector is a viral vector, such as a lentivirus, adenovirus, or adeno-associated virus. A simplified system for generating recombinant adenoviruses is presented, for example, by He T C. et. al. Proc. Natl. Acad. Sci. USA, 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli BJ5183 cells with an adenoviral backbone plasmid, e.g. pAdEasy-1 of STRATAGENE®'s ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, the preferred viral vector is a lentiviral vector and there are many examples of use of lentiviral vectors for gene therapy for inherited disorders of haematopoietic cells and various types of cancer, and these references are hereby incorpaorated by reference (Klein, C. and Baum, C. (2004), Hematol. J., 5:103-111; Zufferey, R et. al. (1997), Nat. Biotechnol., 15:871-875; Morizono, K. et. al. (2005), Nat. Med., 11:346-352; Di Domenico, C. et. al. (2005). Hum. Gene Ther., 16:81-90). The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with VIRAPOWER™ Lentiviral Expression systems from INVITROGEN™ Inc.

In one embodiment, the expression viral vector can be a recombinant adeno-associated virus (rAAV) vector. Using rAAV vectors, genes can be delivered into a wide range of host cells including many different human and non-human cell lines or tissues. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the chimeric DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

Recombinant lentivirus has the advantage of delivery and expression of a nucleic acid encoding an anti-cancer agent or a fusion, or truncated nucleic acid thereof in either dividing and non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with ViraPower™ Lentiviral Expression systems from Invitrogen.

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, one uses of AAV viral vectors comprising a nucleic acid encoding a anti-cancer agent or a fusion, or truncated nucleic acid thereof and/or its variant forms. Recombinant adeno-associated virus (rAAV) vectors are applicable to a wide range of host cells including many different human and non-human cell lines or tissues. Because AAV is non-pathogenic and does not elicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the coding nucleic acid, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

Retroviral vectors are known to the art as useful in delivery of nucleic acid construct and siRNA expression constructs. See, for example, the text of Devroe and Silver (2002), incorporated herein by reference, which discloses that retroviruses are efficient vectors for delivery of siRNA expressing cassettes into mammalian cells. Barton and Medzhitov (2002) disclose that retroviral introduction of siRNA expression constructs results in the stable inactivation of genes in primary cells.

Lentiviruses are a subgroup of retroviruses that can infect nondividing cells owing to the karyophilic properties of their preintegration complex, which allow for its active import through the nucleopore.

Lentiviruses include members of the bovine lentivirus group, equine lentivirus group, feline lentivirus group, ovinecaprine lentivirus group and primate lentivirus group. The development of lentiviral vectors for gene therapy has been reviewed in Klimatcheva et al., (1999). The design and use of lentiviral vectors suitable for gene therapy is described, for example, in U.S. Pat. No. 6,531,123; U.S. Pat. No. 6,207,455; and U.S. Pat. No. 6,165,782 (each specifically incorporated herein by reference). Examples of lentiviruses include, but are not limited to, HIV-1, HIV-2, HIV-1/HIV-2 pseudotype, HIV-1/SIV, FIV, caprine arthritis encephalitis virus (CAEV), equine infectious anemia virus and bovine immunodeficiency virus. HIV-1 are also encompassed for use.

Lentiviral vectors offer great advantages for gene therapy. They integrate stably into chromosomes of target cells which is required for long-term expression. Also, they do not transfer viral genes therefore avoiding the problem of generating transduced cells that can be destroyed by cytotoxic T-cells. Additionally, they have a relatively large cloning capacity, allowing for clinical applicability. Furthermore, lentiviruses, in contrast to other retroviruses, are capable of transducing non-dividing cells. This is very important in the context of gene-therapy for tissues such as the hematopoietic system, the brain, liver, lungs and muscle. For example, vectors derived from HIV-1 allow efficient in vivo and ex vivo delivery, integration and stable expression of transgenes into cells such a neurons, hepatocytes, and myocytes (Blomer et al., 1997; Kafri et al., 1997; Naldini et al., 1996a; 1996b).

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins. Lentiviral vectors are well known in the art, see Naldini et al., (1996a and 1996b); Zufferey et al., (1997); Dull et al. (1998); U.S. Pat. Nos. 6,013,516 and 5,994,136 all incorporated herein by reference. In general, these vectors are plasmid-based or virus-based, and are configured to carry the essential sequences for incorporating foreign nucleic acid, for selection and for transfer of the nucleic acid into a host cell.

Correspondingly, lentiviral vectors derived from human immunodeficiency virus type 1 (HIV-1) can mediate the efficient delivery, integration and long-term expression of transgenes into non-mitotic cells both in vitro and in vivo (Naldini et al., 1996a; Naldini et al., 1996b; Blomer et al., 1997).

In the retroviral genome, a single RNA molecule that also contains all the necessary cis-acting elements carries all the coding sequences. Biosafety of a vector production system is therefore best achieved by distributing the sequences encoding its various components over as many independent units as possible, to maximize the number of crossovers that would be required to re-create an replication competent recombinants (RCR). Lentivector particles are generated by co-expressing the virion packaging elements and the vector genome in host producer cells, e.g. 293 human embryonic kidney cells. In the case of HIV-1-based vectors, the core and enzymatic components of the virion come from HIV-1, while the envelope protein is derived from a heterologous virus, most often VSV due to the high stability and broad tropism of its G protein. The genomic complexity of HIV, where a whole set of genes encodes virulence factors essential for pathogenesis but dispensable for transferring the virus genetic cargo, substantially aids the development of clinically acceptable vector systems.

Multiply attenuated packaging systems typically now comprise only three of the nine genes of HIV-1: gag, encoding the virion main structural proteins, pol, responsible for the retrovirus-specific enzymes, and rev, which encodes a post-transcriptional regulator necessary for efficient gag and pol expression (Dull, et al., 1998). From such an extensively deleted packaging system, the parental virus cannot be reconstituted, since some 60% of its genome has been completely eliminated. In one version of an HIV-based packaging system, Gag/Pol, Rev, VSV G and the vector are produced from four separate DNA units. Also, the overlap between vector and helper sequences has been reduced to a few tens of nucleotides so that opportunities for homologous recombination are minimized.

HIV type 1 (HIV-1) based vector particles may be generated by co-expressing the virion packaging elements and the vector genome in a so-called producer cell, e.g. 293T human enbryonic kidney cells. These cells may be transiently transfected with a number of plasmids. Typically from three to four plasmids are employed, but the number may be greater depending upon the degree to which the lentiviral components are broken up into separate units. Generally, one plasmid encodes the core and enzymatic components of the virion, derived from HIV-1. This plasmid is termed the packaging plasmid. Another plasmid encodes the envelope protein(s), most commonly the G protein of vesicular stomatitis virus (VSV G) because of its high stability and broad tropism. This plasmid may be termed the envelope expression plasmid. Yet another plasmid encodes the genome to be transferred to the target cell, that is, the vector itself, and is called the transfer vector. Recombinant viruses with titers of several millions of transducing units per milliliter (TU/ml) can be generated by this technique and variants thereof. After ultracentrifugation concentrated stocks of approximately 10⁹ TU/ml can be obtained.

The vector itself is the only genetic material transferred to the target cells. It typically comprises the transgene cassette flanked by cis-acting elements necessary for its encapsidation, reverse transcription, nuclear import and integration. As has been previously done with oncoretroviral vectors, lentiviral vectors have been made that are “self-inactivating” in that they lose the transcriptional capacity of the viral long terminal repeat (LTR) once transferred to target cells (Zufferey, et al. 1998). This modification further reduces the risk of emergence of replication competent recombinants (RCR) and avoids problems linked to promoter interference. These vectors, or their components, are known as SIN vectors or SIN containing vectors. The SIN design is described in further detail in Zufferey et al., 1998 and U.S. Pat. No. 5,994,136 both incorporated herein by reference.

Enhancing transgene expression may be required in certain embodiments, especially those that involve lentiviral constructs of the present invention with modestly active promoters. One type of post-transcriptional regulatory element (PRE) is an intron positioned within the expression cassette, which can stimulate gene expression. However, introns can be spliced out during the life cycle events of a lentivirus. Hence, if introns are used as PRE's they may have to be placed in an opposite orientation to the vector genomic transcript.

Post-transcriptional regulatory elements that do not rely on splicing events offer the advantage of not being removed during the viral life cycle. Some examples are the post-transcriptional processing element of herpes simplex virus, the post-transcriptional regulatory element of the hepatitis B virus (HPRE) and the woodchuck hepatitis virus (WPRE). Of these the WPRE is most preferred as it contains an additional cis-acting element not found in the HPRE (Donello et al., 1998). This regulatory element is positioned within the vector so as to be included in the RNA transcript of the transgene, but downstream of stop codon of the transgene translational unit. As demonstrated in the present invention and in Zufferey et al., 1999, the WPRE element is a useful tool for stimulating and enhancing gene expression of desired transgenes in the context of the lentiviral vectors. The WPRE is characterized and described in U.S. Pat. No. 6,136,597, incorporated herein by reference. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7 to 10 fold. Retroviral vectors deliver sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector results in enhanced expression of transgenes.

The introduction of a nucleic acid sequence, such as the nucleic acid sequence encoding the anti-cancer agent into the nucleus of a cell requires importation of the nucleic acids into the nucleus through the nuclear membrane. Lentiviruses utilize an active nuclear import system, which forms the basis of their ability to replicate efficiently in non-dividing cells. This active import system relies upon a complex series of events including a specific modality for reverse transcription. In particular, in HIV-1, the central polypurine tract (cPPT), located within the pol gene, initiates synthesis of a downstream plus strand while plus strand synthesis is also initiated at the 3′ polypurine tract (PPT). After strand transfer of the short DNA molecule, the upstream plus strand synthesis will initiate and proceed until the center of the genome is reached. At the central termination sequence (cTS) the HIV-1 reverse transcriptase is ejected, (released from its template), when functioning in a strand displacement mode. (Charneau, et al., 1994) The net result is a double stranded DNA molecule with a stable flap, 99 nucleotides in length at the center of the genome. This central “flap” facilitates nuclear import. (Zennou, et al., 2000).

The cloned gene for an anti-cancer agent (i.e. a pro-apoptotic gene, cytotoxin, immunotoxin etc) can be manipulated by a variety of well known techniques for in vitro mutagenesis, among others, to produce variants of the naturally occurring human protein, herein referred to as muteins or variants or mutants, which can be used in accordance with the methods and compositions described herein. The variation in primary structure of muteins of an anti-cancer agent (i.e. a pro-apoptotic molecule, cytotoxin molecule and the like), useful in the invention, for instance, can include deletions, additions and substitutions. The substitutions can be conservative or non-conservative. The differences between the natural protein and the mutein generally conserve desired properties, mitigate or eliminate undesired properties and add desired or new properties. For example, in some embodiments, an anti-cancer agent which is a pro-apoptosis molecule can be a pro-apoptotic polypeptide of at least 50 amino acids of the native (i.e. wild type) pro-apoptotic protein or a functional mutein or variant thereof.

In some embodiments, the expressed anti-cancer agent which is a polypeptide (i.e. a cytotoxic molecule, pro-apoptotic molecule and the like) can also be a fusion polypeptide, fused, for example, to a polypeptide that targets the product to a desired location, or, for example, a tag that facilitates its purification, if so desired. Fusion to a polypeptide sequence that increases the stability of an expressed polypeptide, i.e. an expressed anti-cancer polypeptide agent (such as a pro-apoptotic molecule, immunotoxin and cytotoxic molecule etc) is also contemplated. For example, fusion to a serum protein, e.g., serum albumin, can increase the circulating half-life of an anti-cancer polypeptide agent. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use. In another aspect, biodegradable or absorbable polymers can provide extended, often localized, release of polypeptide agents. Such synthetic bioabsorbable, biocompatible polymers, which can release proteins over several weeks or months can include, for example, poly-α-hydroxy acids (e.g. polylactides, polyglycolides and their copolymers), polyanhydrides, polyorthoesters, segmented block copolymers of polyethylene glycol and polybutylene terephtalate (POLYACTIVE™), tyrosine derivative polymers or poly(ester-amides). Suitable bioabsorbable polymers to be used in manufacturing of drug delivery materials and implants are discussed e.g. in U.S. Pat. Nos. 4,968,317 and 5,618,563, which are incorporated herein in their entirety by reference and among others, and in “Biomedical Polymers” edited by S. W. Shalaby, Carl Hanser Verlag, Munich, Vienna, New York, 1994 and in many references cited in the above publications. The particular bioabsorbable polymer that should be selected will depend upon the particular patient that is being treated.

### Use of Engineered ASC Expressing Anti-Cancer Agents for the Treatment of Cancer

One aspect of the present invention provides a method of treatment and/or prophylaxis of a cancer in a subject comprising administering to the subject a composition comprising engineered ASC, where the engineered ASC comprise a nucleic acid sequence encoding an anti-cancer agent.

In some embodiments, the present invention relates to a method of treating a subject with cancer, where a ASC is obtained from a subject, in some embodiments, from the same subject which is to be treated for cancer. The ASC is then genetically modified ex vivo to produce an engineered ASC which expresses at least one anti-cancer agent according to the methods as described herein (i.e. the ASC are transduced ex vivo with a nucleic acid sequence encoding an anti-cancer agent operatively linked to a promoter). In some embodiments, the engineered ASC can be optionally propagated to obtain a substantially pure population of engineered ASC. The engineered ASC are then administered to a subject with cancer. Administration of the engineered ASC which express an anti-cancer agent can be by any suitable means, for example systemic administration or local administration, such as direct injection into the tumor. In preferred embodiments, the engineered ASC is administered intravenously to the vascularization network which supplies blood to the cancer or tumor cell mass.

The use of adipose tissue and adipose-derived regenerative cells (i.e. adipose derived stem cells) for the treatment of cancer has been reported, as disclosed in International Patent Application WO09/076,548 (“548 application”). While the '548 application discusses administering adipose tissue to, or near a tumor site, it does not disclose, suggest or mention the use or administration of a heterogeneous population of adipose-derived stromal cells (comprising at least one of following population of cells; endothelial cells, mesenchymal stem cells, fibroblasts, smooth muscle cells, pericytes or adipose-derived stem cells), as well as additional other cell types not listed to a subject with cancer. Additionally, this '548 application does not discuss or suggest engineering these cells to be used as a delivery tool to deliver anti-cancer agents to a tumor or cancer site in a subject. US Patent Application 2009/0181456 (“456 application”) discusses genetically modified Adipose-derived stem cells (ADSCs) to promote angiogenesis and promote wound closure and for the repair to damaged, degenerated or removed muscle and repair of articular cartilage defects. In particular, this 456 application does not discuss or suggest engineering these cells to express an anti-cancer agent, or in particular an pro-apoptotic agent. Rather, this '456 application discusses the exact opposite, as it discusses engineering stem cells from adipose tissue (ADSC's), not a heterogeneous population of cells derived from adipose tissue (i.e. heterogeneous population of adipose derived stromal cell as disclosed herein), and discusses engineering the homogenous adipose-derived stem cell population with molecules which promote cells growth, including promoting muscle growth in areas of muscle loss due to trauma, vascular insult and tumor resection. This '456 application also discloses expressing anti-angiogenic agents, and thus one of ordinary skill in the art would not use the cells described in the '456 application for the treatment of a subject with cancer. The U.S. Pat. No. 5,980,887 patent ('887 patent) also discusses engineered embryonic-cell derived endothelial cells which express pro- and anti-angiogenic agents to promote growth, in particular to promote endothelial growth of injured blood vessels, in particular to promote angiogenesis in ischemic type disorders. Thus, like the '456 application, the '887 patent discusses promoting cell growth with engineered cells, which is the exact opposite of the present invention which is to inhibit cell growth of cancer cells by engineered cells expressing anti-cancer agents, including but not limited to pro-apoptosis agents.

In some embodiments, the engineered ASC expresses more than one anti-cancer agent, for example, at least 2, or at least 3, or at least 4 or at least 5 or more anti-cancer agents. In some embodiments, where the engineered ASC expresses more than one anti-cancer agent, the anti-cancer agent can be the same type (i.e. polypeptide anti-cancer agents) or different types of agents (i.e. an anti-cancer polypeptide agent and a RNAi anti-cancer agent). In some embodiments, where the engineered ASC expresses more than one anti-cancer agent, the anti-cancer agents can be encoded on the same nucleic acid sequence, (for example separated by IRES sequence) or on a separate nucleic acid sequences. In some embodiments, where the engineered ASC expresses more than one anti-cancer agent, the anti-cancer agents can be operatively linked to the same promoter or a different promoter, for example where different promoters are used, the nucleic acid sequence encoding one anti-cancer agent can be operatively linked to a tissue specific promoter, such as an adipose-tissue promoter, and the nucleic acid sequence encoding a second anti-cancer agent can be operatively linked to in inducible promoter.

Accordingly, in some embodiments, the ASC expressing an anti-cancer agent as disclosed herein is useful for the treatment of cancer. In some embodiments, a cancer, or disease or disorder or malignancy can be any disease of an organ or tissue in mammals characterized by poorly controlled or uncontrolled multiplication of normal or abnormal cells in that tissue and its effect on the body as a whole. In some embodiments, cancers comprise benign neoplasms, dysplasias, hyperplasias as well as neoplasms showing metastatic growth or any other transformations like e.g. leukoplakias which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease. Cancerous cells and tissues can affect the body as a whole when causing paraneoplastic syndromes or if cancer occurs within a vital organ or tissue, normal function can be impaired or halted, with possible fatal results. In some instances, if the function of a vital organ is compromised by cancer or cancer cells, either primary or metastatic, cancer can lead to the death of a subject or mammal affected. A malignant cancer is a cancer which has a tendency to spread and can cause death if not treated. Benign tumors usually do not cause death, although they can lead to death if they interfere with a normal body function by virtue of their location, size, or paraneoplastic side effects.

The ability to administer a population of ASCs to a subject with cancer, where the ASC has been engineered (i.e. nucleic acid has been introduced) to carry an expression vector encoding an anti-cancer agent is useful for the treatment of a variety of benign and malignant cancers (e.g., ascites and solid tumors, carcinomas, leukemias, lymphomas, melanomas, sarcomas) in a subject. Some tumor types of interest are breast, colorectal, lung, ovarian, pancreatic, prostatic, renal, and testicular carcinoma.

Thus, examples of cancers that can be treated with engineered ASC expressing an anti-cancer agent include, for example but are not limited to, small or non-small cell lung, oat cell, papillary, bronchiolar, squamous cell, transitional cell, Walker), leukemia (e.g., B-cell, T-cell, HTLV, acute or chronic lymphocytic, mast cell, myeloid), histiocytoma, histiocytosis, Hodgkin disease, non-Hodgkin lymphoma, plasmacytoma, reticuloendotheliosis, adenoma, adenocarcinoma, adeno-fibroma, adenolymphoma, ameloblastoma, angiokeratoma, angiolymphoid hyperplasia with eosinophilia, sclerosing angioma, angiomatosis, apudoma, branchioma, malignant carcinoid syndrome, carcinoid heart disease, carcinosarcoma, cementoma, cholan-gioma, cholesteatoma, chondrosarcoma, chondroblastoma, chondrosarcoma, chordoma, choristoma, craniopharyngioma, chrondroma, cylindroma, cystadenocar-cinoma, cystadenoma, cystosarcoma phyllodes, dysgerminoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendo-thelioma, hemangioma, hemangiopericytoma, hemangiosarcoma, hepatoma, islet cell tumor, Kaposi sarcoma, leiomyoma, leiomyosarcoma, leukosarcoma, Leydig cell tumor, lipoma, liposarcoma, lymphangioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuro-blastoma, neuroepithelioma, neurofibroma, neurofibromatosis, odontoma, osteoma, osteosarcoma, papilloma, paraganglioma, paraganglioma nonchromaffin, pinealoma, rhabdomyoma, rhabdomyosarcoma, Sertoli cell tumor, teratoma, theca cell tumor, and other diseases in which cells have become dysplastic, immortalized, or transformed.

In some embodiments, the subject to whom a composition comprising engineered ASC which comprise a nucleic acid sequence encoding an anti-cancer agent has not previously had a tumor resection. In some embodiments, a composition comprising engineered ASC as disclosed herein is not administered to promote muscle regeneration in a subject with cancer. In some embodiments, a composition comprising engineered ASC as disclosed herein is not administered at a location or a lesion site where a tumor has been removed from the subject.

Administration of Engineered ASC Expressing an Anti-Cancer Agent to a Subject with Cancer

A population of engineered ASC expressing an anti-cancer agent can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The ASC population may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998). In another aspect, the engineered ASC expressing an anti-cancer agent could be optionally transduced with a gene encoding pro-angiogenic growth factor(s).

Pharmaceutical compositions comprising effective amounts of engineered ASC expressing an anti-cancer agent are also contemplated by the present invention. These compositions comprise an effective number of cells, optionally, in combination with a pharmaceutically acceptable carrier, additive or excipient. In certain aspects of the present invention, cells are administered to the subject in need of a transplant in sterile saline. In other aspects of the present invention, the cells are administered in Hanks Balanced Salt Solution (HBSS) or Isolyte S, pH 7.4. Other approaches may also be used, including the use of serum free cellular media. In one embodiment, the cells are administered in plasma or fetal bovine serum, and DMSO. Systemic administration of the cells to the subject may be preferred in certain indications, whereas direct administration at the site of or in proximity to the diseased and/or site of the tumor may be preferred in other indications or cancer types.

The composition comprising a population of engineered ASC expressing an anti-cancer agent can optionally be packaged in a suitable container with written instructions for a desired purpose, such as the reconstitution of engineered ASC expressing an anti-cancer agent to treat a cancer.

In one embodiment, a composition comprising engineered ASC expressing an anti-cancer agent are administered with a differentiation agent. In one embodiment, the engineered ASC expressing an anti-cancer agent are combined with the differentiation agent to administration into the subject. In another embodiment, the engineered ASC expressing an anti-cancer agent are administered separately to the subject from the differentiation agent. Optionally, if the engineered ASC expressing an anti-cancer agent are administered separately from the differentiation agent, there is a temporal separation in the administration of the ASCs and the differentiation agent. The temporal separation may range from about less than a minute in time, to about hours or days in time. The determination of the optimal timing and order of administration is readily and routinely determined by one of ordinary skill in the art.

### Pharmaceutical Compositions

The composition comprising engineered ASC expressing an anti-cancer agent may be combined with any other drug, in some embodiments another agent for the treatment of the same medicinal indication. For example, a composition comprising a population of engineered ASC expressing an anti-cancer agent may be combined with one or cancer drugs or cancer therapies, for example, surgery, radiation or a chemotherapeutic agent (e.g., such as daunorubicin, idarubicin, mitomycin C, 5-fluorouracil (5-FU), methotrexate (MTX), taxol, vincristine, and cisplatin). Additional suitable teachings for pharmaceutical compositions and their preparation, administration and dosing in relation to oligonucleotide compounds which may be utilized within the scope of the present invention are given in US Patent Application No. 20040146902 which is incorporated herein by reference.

The term “cancer drug” which can be co-administered to a subject with a composition comprising engineered ASC expressing an anti-cancer agent is any agent, compound or entity that would be capably of negatively affecting the cancer in the subject, for example killing cancer cells, inducing apoptosis in cancer cells, reducing the growth rate of cancer cells, reducing the number of mestatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the subject with cancer. Anti-cancer therapy includes biological agents (biotherapy), chemotherapy agents, and radiotherapy agents. The combination of chemotherapy with biological therapy (i.e. administration of a composition comprising engineered ASC expressing an anti-cancer agent) is known as biochemotherapy.

In another embodiment, the cancer therapy includes a chemotherapeutic regimen further comprises radiation therapy. In an alternate embodiment, the therapy comprises administration of an anti-EGFR antibody or biological equivalent thereof.

Chemotherapy and therapeutic anticancer agents or cancer drugs which can be used in conjunction (either simultaneously, prior to or following) administration of engineered ASC cells to the subject include, cytotoxic agents such as Taxol, Cytochalasin B, Gramicidin D, Ethidium Bromide, Emetine, Mitomycin, Etoposide, Tenoposide, Vincristine, Vinblastine, camptothecin (CPT), Colchicin, Doxorubicin, Daunorubicin, Mitoxantrone, Mithramycin, Actinomycin D, 1-Dehydrotestosterone, Glucocorticoids, Procaine, Tetracaine, Lidocaine, Propranolol, and Puromycin and analogs or homologs thereof. Therapeutic agents include, but are not limited to, antimetabolites (e.g., Methotrexate, 6-Mercaptopurine, 6-Thioguanine, Cytarabine, 5-Fluorouracil, Decarbazine), alkylating agents (e.g., Mechlorethamine, Thiotepa, Chlorambucil, Melphalan, Carmustine (BCNU), Lomustine (CCNU), Cyclophosphamide, Busulfan, Dibromomannitol, Streptozotocin, Mitomycin C, Cis-Dichlorodiamine Platinum (II) (DDP), Cisplatin), anthracyclines (e.g., Daunorubicin (formerly Daunomycin) and Doxorubicin), antibiotics (e.g., Dactinomycin (formerly Actinomycin), Bleomycin, Mithramycin, and Anthramycin (AMC)), anti-mitotic agents (e.g., Vincristine and Vinblastine) and selective apoptotic agents such as APTOSYN® (Exisulind), PANZEM® (2-methoxyestradiol), and VELCADE® (bortezomib) a proteasome inhibitor.

Exemplary anticancer agents for the treatment of ovarian cancer can include one or more of the following: Etoposide, Melphalan, Cisplatin, Carboplatin, CPT, Paclitaxel, Anthracyclines (e.g., Doxorubicin), Hexamethylamine (Altretamine), Progestins (e.g., Medroxyprogesterone acetate, Megestrole acetate), 5-Fluorouracil plus Leucovorin (to counteract folic acid antagonists), Ifosfamide, or Topotecan.

Exemplary anticancer agents for the treatment of breast cancer can include Doxorubicin, PANZEM® (2-methoxyestradiol), Paclitaxel, Methotrexate, 5-Fluorouracil, Docetaxel, Thiotepa, Cisplatin, Estrogen receptor modulators such as Tamoxifen and Toremifene, Estrogens (e.g., diethylstilbestrol), Androgens (e.g., fluoxymesterone), Gonadotropin-Releasing Hormone (GnRH), Anastrozole, Aromatase inhibitors (antineoplastics), Vinorelbine tartrate, Gemcitabine hydrochloride, Progestins (e.g., Medroxyprogesterone acetate, Megestrole acetate), Trastuzumab (HERCEPTIN®), and Cyclophosphamide. Anticancer agents for colorectal cancer treatment can include Oxaliplatin, 5-Fluorouracil, or Leucovorin.

Exemplary anticancer agents for the treatment of prostate cancer can include anti-androgens (e.g., Flutamide, Nilutamide, Bicalutamide, Cyproterone, Megestrol) and the Leuteinizing Hormone-Releasing Hormone analogues (e.g., Buserelin, Goserelin, Leuprolide). Anticancer agents for liver cancer treatment can include 5-Fluorouracil, Leucovorin, Raltitrexed, Mitomycin C, and CPT-1. Anticancer agents for the treatment of lung cancer can include Paclitaxel, Carboplatin, Vinorelbine tartrate, Gemcitabine hydrochloride, Etoposide, Doxorubicin, Ifosfamide, Docetaxel, Cyclophosphamide, Methotrexate, Lomustine (CCNU), Topotecan hydrochloride, and Cisplatin.

In some embodiments, the cancer treatment comprises the administration of the genetically engineered adult ASCs and a chemotherapeutic drug selected from the group consisting of fluoropyrimidine (e.g., 5-FU), oxaliplatin, CPT-11, (e.g., irinotecan) a platinum drug or an anti EGFR antibody, such as the cetuximab antibody or a combination of such therapies, alone or in combination with surgical resection of the tumor.

In yet a further aspect, the treatment compresses radiation therapy and/or surgical resection of the tumor masses in conjunction with administering the genetically engineered adult ASCs into the tumor or the location where the main tumor mass has been removed from. In one embodiment, the present invention encompasses administering to a subject identified as having, or increased risk of developing RCC an anti-cancer combination therapy where combinations of anti-cancer agents are used, such as for example TAXOL, cyclophosphamide, cisplatin, gancyclovir and the like. Anti-cancer therapies are well known in the art and are encompassed for use in the methods of the present invention. Chemotherapy includes, but is not limited to an alkylating agent, mitotic inhibitor, antibiotic, or antimetabolite, anti-angliogenic agents etc. The chemotherapy can comprise administration of CPT-11, temozolomide, or a platin compound. Radiotherapy can include, for example, x-ray irradiation, w-irradiation, β-irradiation, or microwaves.

In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors and gliomas include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex or Trexall), irinotecan (CPT-11); erlotinib; oxalipatin; anthracyclins-idarubicin and daunorubicin; doxorubicin; alkylating agents such as melphalan and chlorambucil; cis-platinum, methotrexate, and alkaloids such as vindesine and vinblastine.

In one embodiment, the pharmaceutical agents comprising nucleic acid construct of the invention can be administered to subjects. Examples of subjects include mammals, e.g., humans and other primates; cows, pigs, horses, and farming (agricultural) animals; dogs, cats, and other domesticated pets; mice, rats, and transgenic non-human animals.

This invention is further illustrated by the following examples which should not be construed as limiting. The contents of all references cited throughout this application, as well as the figures and tables are incorporated herein by reference. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### REFERENCES

The references, including articles, patents and patent applications cited in the specification are all incorporated herein in their entirety by reference.

1. A method for the treatment of cancer in a subject, the method comprising administering to the subject a composition comprising including an isolated population of adipose-derived stromal cells (ASC) derived from adipose tissue, wherein at least one ASC in the isolated population of ASC comprises a first nucleic acid sequence operatively linked to a first promoter, wherein the first nucleic acid sequence encodes at least one anti-cancer agent, and wherein expression of the first nucleic acid sequence encoding at least one anti-cancer agent is for the treatment of cancer.

2. The method of claim 1, wherein the isolated population of ASC is obtained from the same subject to which the composition is administered.

3. The method of claim 1, wherein the anti-cancer agent is selected from the group consisting of nucleic acid, protein, peptide, siRNA, antisense nucleic acid, asRNA, RNAi, miRNA, antibodies and Fc fragments and combinations thereof.

4. The method of claim 1, wherein the composition comprising the population of adipose-derived stromal cells (ASC) is administered systemically or locally to the tumor.

5. The method of claim 1, wherein the anti-cancer agent is an pro-apoptotic molecule or a pro-apoptotic fragment thereof.

6. The method of claim 1, wherein the anti-cancer agent is not an anti-angiogenic or a pro-angiogenic agent.

7. The method of claim 1, wherein the first nucleic acid sequence encoding at least one anti-cancer agent also encodes a secretory sequence.

8. The method of claim 1, wherein the ASC further comprises a second nucleic acid sequence operatively linked to a second promoter, wherein the second nucleic acid encodes at least one cell death gene.

9. The method of claim 1, wherein the subject is a mammalian subject.

10. The method of claim 9, wherein the mammalian subject is a human subject.

11. The method of claim 9, wherein the mammalian subject has not had a resection of a tumor.

12. The method of claim 1, wherein the administration of said composition comprising an isolated population of ASC derived from adipose tissue is not administered to a location where a tumor has been excised from a subject.

13. The method of claim 1, wherein the ASC is transfected with the first nucleic acid sequence operatively linked to a first promoter ex vivo.

14. The method of claim 1, wherein the anti-cancer agent is a polypeptide.

15. The method of claim 14, wherein the polypeptide is a protein which activates a pro-drug.

16. The method of claim 1, wherein the anti-cancer agent is an inhibitor to an oncogene.

17. The method of claim 16, wherein the inhibitor to an oncogene is a RNAi molecule to the oncogene.

18. The method of claim 16, wherein an oncogene is selected from the group of cancer genes consisting of: HER2/Her-2, BRAC1 and BRAC2, Rb, p53, and variants thereof.

19. The method of claim 17, wherein an oncogene is selected from the group of cancer genes consisting of: HER2/Her-2, BRAC1 and BRAC2, Rb, p53, and variants thereof.

20. The method of claim 1, wherein the cancer is a solid tumor.
